(19) United States
(12) Patent Application Publication
(10) Pub. No.: US 2025/0144427 A1
(43) Pub. Date: May 8, 2025

(54) PREDICTING AND MANAGING SEIZURES IN AN INDIVIDUAL USING BRAIN IMPEDANCE MEASUREMENTS

(71) Applicant: Mayo Foundation for Medical Education and Research, Rochester, MN (US)

(72) Inventors: Gregory A. Worrell, Rochester, MN (US); Vaclav Kremen, Rochester, MN (US); Benjamin H. Brinkmann, Byron, MN (US); Vladimir Sladky, Rochester, MN (US); Filip Mivalt, Rochester, MN (US); Jie Cui, Rochester, MN (US)

(21) Appl. No.: 18/821,702

(22) Filed: Aug. 30, 2024

Related U.S. Application Data
(60) Provisional application No. 63/595,739, filed on Nov. 2, 2023.

(51) Int. Cl. A61N 1/36; A61B 5/00; A61B 5/053

(52) U.S. Cl. CPC A61N 1/3614; A61B 5/053; A61B 5/4094; A61B 5/4809; A61B 5/4839; A61B 5/7267; A61B 5/7275; A61B 5/746; A61N 1/36064

(57) ABSTRACT

Systems and methods for predicting and managing seizures in an individual are disclosed. In one aspect, a method for predicting and managing seizures in an individual. In another aspect, a system comprising an implanted device configured to capture at least one measurement of an impedance of brain tissue of an individual and a computing device configured to. in response to a prediction that the individual is likely to experience a seizure, initiate a remedial action that comprises at least one of alerting the individual or another user that the individual is likely to experience the seizure, applying an electrical stimulus to the brain tissue of the individual to reduce a likelihood of the seizure occurring or to reduce a severity of the seizure, or delivering a drug to the brain tissue of the individual to reduce a likelihood of the seizure occurring or to reduce a severity of the seizure.

### CROSS-REFERENCE TO RELATED APPLICATIONS

This application claims the benefit of priority under 35 U.S.C. § 119(e) to U.S. provisional application Ser. No. 63/595,739 filed Nov. 2, 2023, the entire contents of which are incorporated by reference herein.

### STATEMENT AS TO FEDERALLY SPONSORED RESEARCH

This invention was made with government support under NS095495 awarded by the National Institutes of Health. The government has certain rights in the invention.

### BACKGROUND

Brain tissue can be stimulated using wireless implants that comprise flexible substrates and electronics thereon, and include stimulating electrodes and recorder circuitry for monitoring brain activity.

People with epilepsy face the challenge of unpredictable seizures, significantly affecting their daily life quality. Even those resistant to medication spend less than 1% of their time experiencing a seizure, while the remainder is spent under constant seizure threat. Currently, people with epilepsy rely on daily anti-seizure medications that often have short-lived effects.

### SUMMARY

This document describes systems, methods, devices, and other techniques used for seizure detection, intervention, and prevention.

One aspect is a method for predicting and managing seizures in an individual, the method comprising obtaining at least one measurement of an impedance of brain tissue of the individual to determine an impedance signature of the brain tissue of the individual, processing the impedance signature of the brain tissue to predict whether the individual is likely to experience a seizure, and in response to predicting based on the impedance signature that the individual is likely to experience a seizure, initiating a remedial action that comprises at least one of alerting the individual or another user that the individual is likely to experience the seizure, applying an electrical stimulus to the brain tissue of the individual to reduce a likelihood of the seizure occurring or to reduce a severity of the seizure, or delivering a drug to the brain tissue of the individual to reduce a likelihood of the seizure occurring or to reduce a severity of the seizure.

Another aspect is a system comprising an implanted device configured to capture at least one measurement of an impedance of brain tissue of an individual and a computing device configured to obtain, from the implanted device, the at least one measurement of the impedance of the brain tissue of the individual and in response to a prediction that the individual is likely to experience a seizure, initiate a remedial action that comprises at least one of alerting the individual or another user that the individual is likely to experience the seizure, applying an electrical stimulus to the brain tissue of the individual to reduce a likelihood of the seizure occurring or to reduce a severity of the seizure, or delivering a drug to the brain tissue of the individual to reduce a likelihood of the seizure occurring or to reduce a severity of the seizure, wherein the prediction that the individual is likely to experience a seizure is determined by processing an impedance signature determined from the at least one measurement of the impedance of the brain tissue of the individual.

### DESCRIPTION OF DRAWINGS

FIG. 1 illustrates an example environment for monitoring a patient and providing an alert in response to predicting that a seizure is likely to occur.

FIG. 2 illustrates an example method for providing an alert when it is predicted that a seizure is likely to occur.

FIG. 3A presents the impedance levels and rate of change within a ±5-min window surrounding seizure onset, compiled from five subjects.

FIG. 3B shows the impedance levels and rate of change within a ±12-hours window surrounding seizure onset, compiled from five subjects.

FIG. 4 illustrates an example seizure forecasting system.

FIG. 5A illustrates an EPA system that enables synchronized bidirectional communications between patient, implantable neural sensing, stimulation devices, and local and/or distributed cloud computing infrastructure.

FIG. 5B illustrates continuous streaming of brain LFP enables monitoring of epileptiform activity [IES and seizure (Sz)] and Z.

FIG. 5C illustrates representative raw impedance time series from HPC, AMG, and ANT recorded over multiple months from the subject living in natural home environment.

FIG. 6 illustrates a table presenting patient demographics and clinical information.

FIG. 7 illustrates a table presenting electrode coregistration.

FIG. 8 illustrates a table presenting automated seizure detection and review results.

FIG. 9 illustrates a table presenting sleep-wake classification scoring accuracy.

FIG. 10A illustrates a benchtop phantom fixture was used for testing two- and four-point impedance measurements in saline/microbead composites

FIG. 10B illustrates a graph presenting 2- and 4-point impedance measurements.

FIG. 10C illustrates graphs for RC+S measured impedance value and measured traces.

FIG. 11 illustrates a sleep characteristics table.

FIG. 12A illustrates a circuit model for monopolar brain impedance measurements.

FIG. 12B illustrates example impedance traces from electrodes targeting HPC, AMG, and ANT anatomic locations within the first 4 weeks (˜30 d) post-implantation.

FIG. 12C illustrates normalized stable impedance for the anatomic locations.

FIG. 13A illustrate circular histograms illustrating the distribution of seizure onset times and sleep periods over a 24 h clock for each subject.

FIG. 13B illustrate impedance measurements from AMG, ANT, and HPC during the ±12 h surrounding spontaneous seizures were compared with days without seizures.

FIG. 14A illustrate circular histograms of representative of 24 h impedance values.

FIG. 14B illustrate the group-level composite polar plots from HPC, ANT, and AMG averaged across all subjects demonstrates that the min/max impedance values over 24 h periods of day/night are not uniformly distributed.

FIG. 14C illustrate composite polar plots for individual subjects M1-5 over multiple months show the average impedance increases during wakefulness and decreases during sleep.

FIG. 15 illustrate a table representing circadian impedance cycles for HPC, AMG, and THL in individual patients.

FIG. 16A illustrates impedance time series and analysis.

FIG. 16B illustrates a blowup of CWT.

FIG. 16C illustrates statistical testing using Thomson F test multitaper scheme to identify significant ultradian, circadian, and infradian impedance cycles.

FIG. 16D illustrates box plots of percentage of significant impedance cycles in ANT, AMG, and HPC.

FIG. 17A illustrates impedance changes over a single night during a hospital stay with gold standard sleep-wake classifications from polysomnography, including wakefulness and four cycles of NREM and REM sleep from subject M5.

FIG. 17B illustrates group-level analysis taken from impedance measurements during sleep.

FIG. 17C illustrates group level analysis taken from impedance measurements during daytime naps.

FIG. 17D illustrates, at the individual subject level, the ANT, AMG, and HPC impedance is greater during wakefulness compared with NREM sleep in all subjects, and it is greater than REM.

FIG. 18 illustrates a table for impedance changes during sleep-wake behavioral state transitions.

FIG. 19 illustrates a graph presenting four-point impedance measurement in a μbead-saline composite.

FIG. 20 illustrates a graph presenting an impedance (linear response) in saline, gelatine, saline-gelatine-μbead electrolytes, and in-vivo pig.

FIG. 21 illustrates graphs of measured impedance in human subjects.

FIGS. 22A-C illustrate graphs for four-point measured impedance in in-vivo pig.

FIG. 23 shows an example of a computing device and a mobile computing device that can be used to implement the techniques described herein.

Like reference symbols in the various drawings indicate like elements.

### DETAILED DESCRIPTION

This specification generally describes systems, methods, devices, and other techniques used for seizure detection, intervention, and prevention. In some implementations, systems and methods for brain impedance sensing for seizure detection, intervention, and prevention are disclosed. In some examples, brain impedance is periodically sampled at one or more locations over a long-term period. The detected impedance dynamics information is integrated into a system for seizure detection and forecasting. In some implementations, deep-learning models are used. In some implementations, adaptive therapy techniques are implemented and applied.

In some examples, the mechanisms underlying changes in extracellular space, impedance dynamics, and seizure generation are analyzed. Impedance measurements are then applied as measurements are detected to make seizure predictions. For example, to make predictions in real-time or near-real time.

In some aspects, systems and methods using brain impedance for seizure forecasting are disclosed. As described below, impedance has a circadian cycle. In some of the seizure forecasting methods disclosed herein, detected deviations from an individual patient's impedance circadian rhythm is used independently or in conjunction with other data input. Examples of other data input include iEEG data including spike detection, high frequency oscillations, or evoked potentials (induced by electrical or sensory stimulus). In some implementations, impedance measurements are used for sleep staging, clearing extracellular protein aggregates, and seizure detection and forecasting. In some implementations, data from wearables, data from implanted EEG, subscalp EEG, or scalp EEG (e.g. evoked potentials, high frequency oscillations, local field potentials, spike detection) and impedance can be combined into a holistic, real-time monitoring of brain excitability and seizure risk.

In some examples, impedance of brain tissue is measured using 2- or 4-point measurements. In some implementations, the measurements are taken local to a location where the electrical stimulation is applied. In some implementations, the measurements are taken from tissue that is not local to where the electrical stimulation is applied. For example, the network response may be measured from stimulation at one node and measured potential at a different node.

In some implementations, macro (mm scale), meso (0.1 mm scale), and/or micro (0.001 mm scale) electrodes are used to stimulate and record from contacts. In some implementations, a wide dynamic range for stimulations and sensing is used. In some particular examples the wide dynamic range includes values from 0.01 to 10,000 Hz. However, other values may also be used. In some implementations, the sampling frequency ranges from 100 Hz to 40,000 Hz for sensing. Other ranges and values can also be used. A range of stimulation currents for impedance can be evoked. For example, a range of stimulation currents from 0.05 to 5 mA can be used in some examples (other ranges are also possible). Additionally or alternatively, different stimulation paradigms can be applied. For example, white noise stimulation, sinusoidal current, square pulses, etc. In some examples the square pulses can range from 10 μs to 1000 μs. Any combination of the above can be used in different implementations, including dynamic implementations which selects values that are most accurately tuned to the implantable device and/or the patient.

In some implementations, the impedance of brain tissue is monitored over time a patient specific threshold or model is used to determine if a seizure is likely to occur. For example, a current impedance (e.g., from one reading a series of readings) can be compared to a longer term mean. An unexpected difference (e.g., a change not caused by change in brain state) is indicative that a seizure may occur. In some implementations, the threshold is a change in impedance value. In some of these implementations the threshold or model is further based on circadian rhythm. In one example, method brain state and impedance are monitored and changes in impedance that are anomalous to the current brain stare are indicative that a seizure is likely to occur. In some implementations, the threshold or model is further based on characteristics of the pulse used to stimulate the brain tissue for measuring tissue. For example, the pulse may use a square wave, sinusoidal wave, white noise simulation, etc. In some implementations, the threshold or model further consider a rate in change of impedance. In some examples, the velocity of the rate of change of impedance increases prior to a seizure. In some implementations, different time scales of impedance values are considered and compared. For example, impedance measurements collected over seconds, minutes, hours, days, weeks, months, years, etc. may considered and compared to detect patterns and features that are indicative of a user being likely to experience a seizure.

In some implementations, a patient specific classifier is built. In one example, an implantable device is implanted on a patient's brain tissue, where it continuously records impedance values that are used as training data for the patient specific classifier. These readings set baselines e.g., for expected sleep and wake state impedances as well as features observed before, after, and during a seizure. In some implementations, a patient self-records when a seizure occurred. In other implementations, impedance readings are processed to determine when a seizure may have occurred. In other implementations, further data is detected and processed to determine whether a seizure occurred. For example, other device may be used to measure brain activity. Various combinations of the data described above can be used to train the patient specific classifier. In some implementations, continuous learning techniques are used to further train the patient specific classifier or other classifier and/or deep learning models described herein.

In some implementations, the systems and method disclosed herein are used for spatial and temporal forecasting of seizures. In some implementations, the classifier disclosed herein predict when a seizure is going to occur and where in the brain the seizure is going to occur. In some implementations, the systems and methods disclosed herein are used to notify a user that the user has gone from a high risk of seizure state back to a low risk of seizure state. In some implementations, a patient in a high risk state is instructed to take a medication or another type of treatment may be applied. In other examples, the patient is instructed to put themselves in a safe location and position for experiencing a seizure.

In some implementations, systems and methods for electrical stimulation and linear response for tracking and modulating extracellular space and brain excitability are used.

Electrical stimulation and associated measurement of linear response (impedance) of tissue (e.g., brain tissue) is used to track and modulate the tissue extracellular space and excitability in a human brain. Impedance is a fundamental electrical property of brain tissue and important for understanding local field potentials and electrical stimulation. More specifically, brain impedance characterizes the resistance to ionic current flow through brain extracellular, intracellular, and vascular compartments.

In some implementations, brain impedance is used to measure the volume of the extracellular space; and then using the impedance measurements to guide manipulation of the size of the extracellular space therapeutically by stimulating brain tissue through the activation the glymphatic system based upon the brain impedance measurements, wherein the amount of brain impedance can be indicative of when to apply electrical stimulation to active the glymphatic system, which in turn sends fluid through the extracellular space to increase the size and volume of the extracellular space to wash away negative brain impurities. In some implementations, brain impedance measurements are used to determine the various levels of sleep stages a patient has undergone by measuring the glymphatic stages of the brain to determine how much deep sleep was done. In some examples, tying brain impedance measurements to sleep stages (and the lack thereof of sleep) and activating the glymphatic system to increase extracellular space at specified times, seizures can be predicted and prevented.

System and methods for classifying and modulating brain behavioral states are discussed in U.S. Pat. No. 11,647,962, entitled “SYSTEM AND METHOD FOR CLASSIFYING AND MODULATING BRAIN BEHAVIORAL STATES”, the disclosure of which is hereby incorporated by reference in its entirety. Materials and methods for using electrical stimulation to treat a mammal having a proteinopathy (e.g., neurodegenerative diseases) or at risk of developing a proteinopathy are discussed in U.S. Pat. No. 11,426,577, entitled “NEUROMODULATION TO MODULATE GLYMPHATIC CLEARANCE”, the disclosure of which is hereby incorporated by reference in its entirety. Therapy systems with quantified biomarker targeting, including for epilepsy treatment, and associated systems and methods are discussed in U.S. Patent Publication Number 20210346809, entitled “THERAPY SYSTEMS WITH QUANTIFIED BIOMARKER TARGETING, INCLUDING FOR EPILEPSY TREATMENT, AND ASSOCIATED SYSTEMS AND METHODS”, the disclosure of which is hereby incorporated by reference in its entirety.

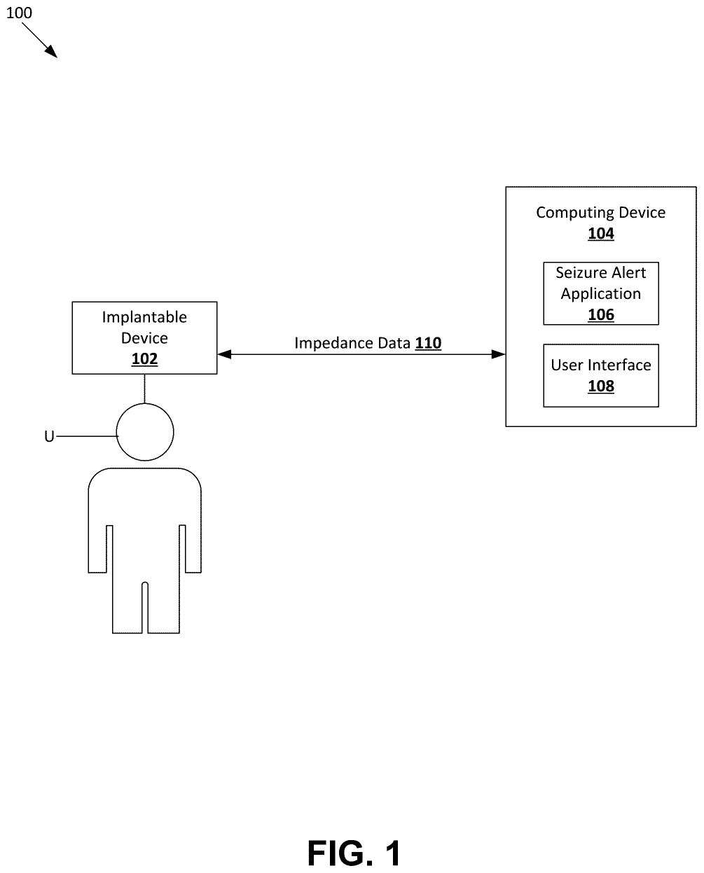

FIG. 1 illustrates an example environment 100 for monitoring a patient and providing an alert in response to predicting that a seizure is likely to occur. The environment 100 includes a user U (e.g., a patient) with an implantable device 102. The implantable device 102 is in wireless communication with a computing device 104 to provide impedance data 110 measured at the implantable device. The computing device 104 includes a seizure alert application 106 and a user interface 108.

The implanted device 102 is configured to capture impedance data 110 of brain tissue of the user U. The implanted device 102 comprises a plurality of electrodes that are surgically implanted in various locations with the user's brain. The implanted device 102 is configured to sample a brain's impedance by injecting current into the brain tissue and sensing the corresponding voltage signals. Further examples of implanted devices 102 are disclosed herein. In some implementations, the implanted device 102 is configured to continuously measure brain tissue. In some implementations, the brain tissue is densely sampled using the implanted device 102.

In some examples, the implantable device 102 is configured to interface with a cloud computing environment. For example, via the computing device 104 or other edge computing devices.

The computing device 104 comprises a user interface 108, and is configured to receive the impedance data 110 from the implanted device 102 and to present an alert via the user interface 108 based on a prediction that the user U (for example, a user associated with the computing device) is likely to experience a seizure, wherein the prediction is based on an impedance signature derived from the impedance data 110.

The seizure alert application 106 is an application configured to alert (e.g., via broadcasting visual and/or audio alarms) the user U when a seizure is predicted as likely to occur. In some implementations, the seizure alert application is configured to relay information from the implanted device to the cloud. In some implementations, the seizure alert application 106 is an application configured to receive a user input for indicating that the user is experiencing a seizure or recently experienced a seizure. In some of these implementations, the user input is used to determine when the user experience a seizure and impedance data from around the time the seizure occurred is collected and processed to determine an impedance signature that corresponds to the user being likely to experience a seizure.

In some implementations, the seizure alert application 106 runs a deep learning model to forecast whether a seizure is likely to occur at the computing device 104. For example, when the computing device 104 has sufficient computing capacity to run the deep learning model. In other examples, the seizure alert application 106 interfaces with a cloud computing environment to run the deep learning model.

In some implementations, the computing device 104 is a mobile computing device such as a tablet, smart phone, smart watch, etc. In some implementations, the computing device 104 is a specialized computing device designed and configured for seizure monitoring. In some implementations, the computing device 104 and the implanted device 102 are configured to allow bi-directional wireless streaming.

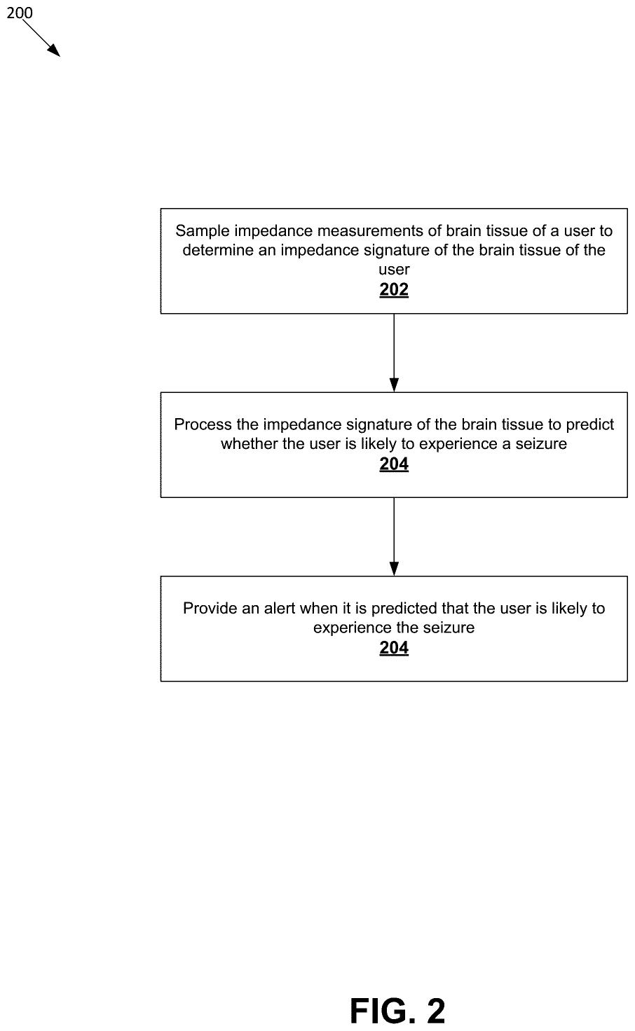

FIG. 2 illustrates an example method 200 for providing an alert when it is predicted that a seizure is likely to occur. The method 200 includes the operations 202, 204, and 206.

The operation 202 samples impedance measurements of brain tissue of a user to determine an impedance signature of the brain tissue of the user. In some implementations, sampling impedance measurements of brain tissue of a user is performed by, injecting an electrical current into the brain tissue with an implanted device, and sensing a voltage in the brain tissue with the implanted device in response to the applied electrical stimulation, and calculating impedance based on the electrical current and sensed voltage. In some implementations, the impedance signature includes an absolute value of observed impedance and a rate of change in impedance.

The operation 204 processes the impedance signature of the brain tissue to predict whether the user is likely to experience a seizure. In some of these implementations, an absolute value of observed impedance and/or a rate of change in impedance are compared to threshold values to predict whether a user is likely to experience a seizure. In some implementations, the thresholds are calibrated for a specific patient. In some implementations, a seizure is predicted as being likely to occur based on a detected a change in impedance of the brain tissue.

In some implementations, a deep learning model is used to predict whether the user is likely to experience a seizure. In some of these implementations, the primary architecture of the deep learning model is a bi-directional long-term memory model. In some implementations, training the machine learning model comprises aggregating model inputs by segmenting impedance data and determining rate of impedance change and time-frequency power signals for each segment, processing the model inputs with a parallel 1-D convolution network (CNN) to extract features, constructing feature representations to learn valid information through forward and/or backward propagation of the extracted features, applying an attention mechanism to redistribute weights of the feature representations to emphasize critical features of preictal stage; and train a full connection layer to output a preictal probability based on the feature representations with redistributed weights. In some examples, the preictal probability is compared to a threshold to determine whether a seizure is likely to occur. In some implementations, the deep learning model is trained using a cloud computing system.

In some implementations, the prediction of whether the user is likely to experience a seizure is further based on a detected brain state. In some of these implementations, the brain states include an awake state and one or more sleep states. Examples of sleep states include REM state, NREM state, etc. In some implementations, the method 200 further comprises measuring glymphatic stages of the brain tissue based on the impedance signature and determining levels of sleep stages for the patient based on the measured glymphatic stages.

The operation 206 provides an alert when it is predicted that the user is likely to experience the seizure. In some implementations, the alert is a visual and/or audio alter that is presented on a user computing device of the patient.

In some implementations, the method 200 further comprises applying a second electrical stimulation to manipulate an extra cellular space in the brain tissue in response to predicting that the seizure is likely to occur.

In some implementations, the operation 206 results in other actions, in addition to or instead of providing an alert. In some implementations, a treatment is applied or altered based on the prediction of whether a seizure will occur. Some implementations include providing an alert to patient alert the patient of a high risk period to allow the patient administers a prophylactic medication. In some implementations, an alert is provided to an implanted neurostimulator (e.g., the implanted device 102 shown in FIG. 1) and the implanted neurostimulator applies and/or adjusts a stimulation. Some implementations, include combinations thereof.

In some implementations, the user is altered that they are likely to have a seizure within some period of time (i.e. 1 minute, 30 minutes, 3 hours). In some implementations, a user is alerted if they are at higher risk or lower risk of seizure that day than what is typical for that patient. In some implementations, a user is alterted to a higher or lower risk period in their infradian seizure cycle. Some implementations, include combinations thereof.

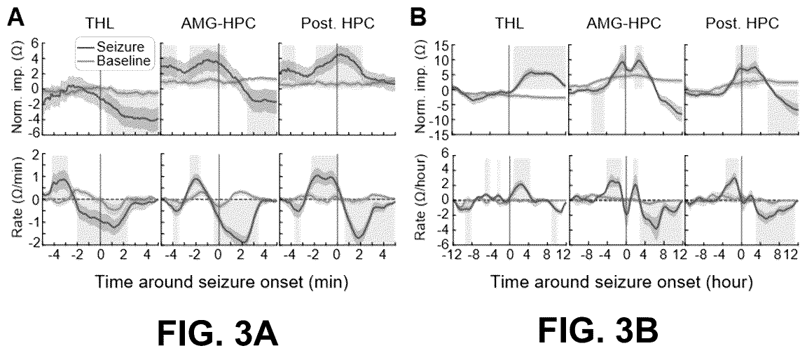

FIGS. 3A and 3B present analysis of impedance dynamics related to seizures. FIG. 3A presents the impedance levels and rate of change within a ±5-min window surrounding seizure onset (indicated by red vertical lines), compiled from five subjects. The top row illustrates the impedance level associated with actual seizures (blue) and baseline events (green, time-matched seizure-free epochs) in the three anatomical locations under investigation: the Thalamus (THL), Amygdala-Hippocampus (AMG-HPC), and Posterior Hippocampus (Post. HPC). Shaded regions denote significant difference between the actual and baseline impedance levels, with lower-half shading indicating lower levels than baseline epoch impedance levels, and upper-half shading indicating higher impedance levels. The bottom row depicts the rate of impedance change associated with actual seizures (blue) and baseline time-matched seizure free epochs (green). FIG. 3B shows the impedance levels and rate of change within a ±12-hours window surrounding seizure onset. In both FIGS. 3A and 3B, blue and green error bands indicate ±3 SEM, where the following abbreviations are used: Norm., Normalized; imp., impedance; THL, thalamus; AMG-HPC, Amygdala-Hippocampus; Post. HPC, Posterior Hippocampus.

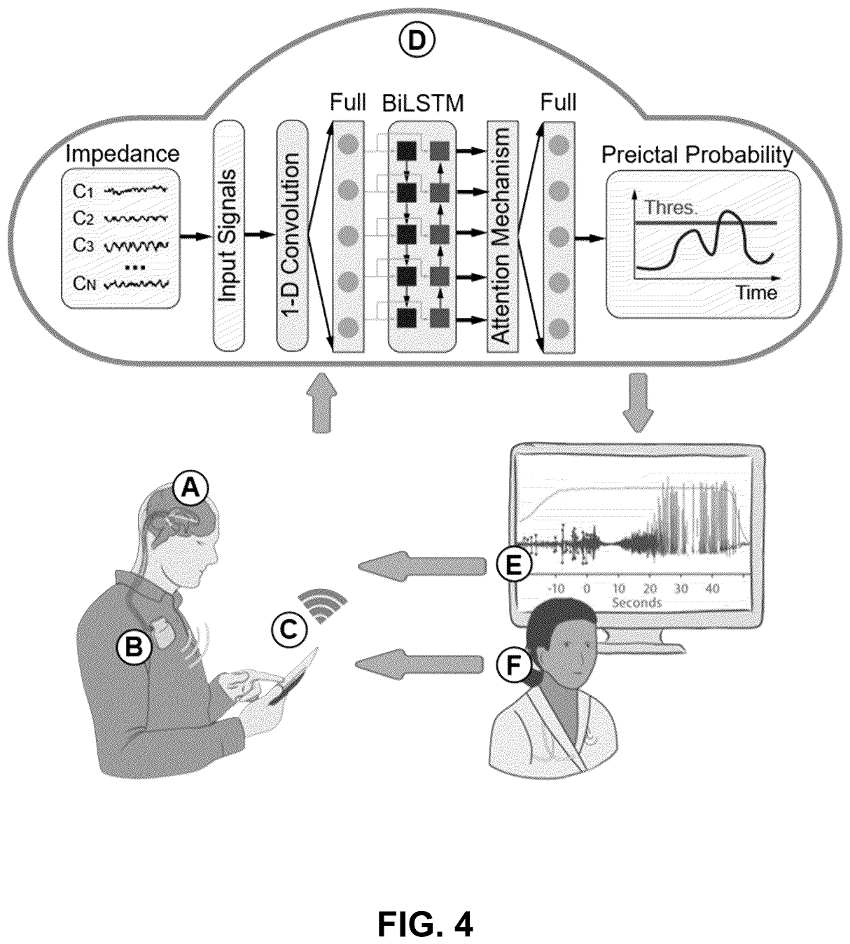

FIG. 4 illustrates a schematic diagram of the seizure forecasting system based on impedance dynamics. The system operates in a closed-loop manner, with impedance periodically sampled via (A) implanted electrodes at multiple brain locations over an extended period. The measurement process is managed by the (B) implanted device, which wirelessly transmits the measurements to the patient's (C) Electronic Personal Assistance Device (EPAD). The EPAD then streams the data to the (D) cloud co-processor via the internet, where a deep-learning model (described herein) computes the probability of predicted seizure risk at each time instance. The estimated seizure risk and other related information are sent back to (E) the monitoring station at a designated medical center, where (F) a medical/research team can check critical patient information in real-time. Seizure risk information can be automatically fed back to (C) the patient's handheld device, or a physician can quickly review the automatic estimation and decide whether to send an alarm to the patient. Upon receiving the seizure risk warning, the EPAD will broadcast audio and visual alarms to the patient. Optionally, the EPAD can send signals to the (B) implanted device to initiate preventive deep brain or cortical therapeutic stimulation through the (A) implanted electrodes. The integrated system provides comprehensive seizure monitoring, forecasting, and adaptive neuromodulation therapy with high performance. The following abbreviation is used in FIG. 4: Thres., threshold.

### Example Implementation 1

People with epilepsy (PWE) face the challenge of unpredictable seizures, significantly affecting their daily life quality. Even those resistant to medication spend less than 1% of their time experiencing a seizure, while the remainder is spent under constant seizure threat. PWE rely on daily anti-seizure medications that often have side effects. Seizure forecasting can transform epilepsy therapy and the lives of PWE by allowing preemptive administration of medications and electrical brain stimulation. A closed-loop system that integrates brain sensing, distributed computing, and neuromodulation therapy for seizure detection, intervention, and prevention is disclosed herein. As an integral component of the system, brain impedance is periodically sampled at multiple locations over a long-term period. Impedance has a close relationship with the brain's extracellular space (ECS), which plays a critical role in influencing neuronal excitability and seizures. There is a correlation between periodic changes in ECS, impedance oscillations, and behavioral state in human limbic system. A significant increase in impedance approximately 1-3 hours before a seizure is a useful biomarker indicative of increased seizure likelihoods. By integrating the impedance dynamics information into the system an automatic system for high performance seizure forecasting using deep-learning model and adaptive therapy with is implemented.

In some implementations, a deep-learning model for seizure forecasting is developed and applied. The deep learning model leverages impedance tracking and other biomarkers for automatic seizure prediction.

In some implementations, a clinically viable closed-loop neuromodulation system for seizure prediction, detection, and therapeutic intervention is developed. In some implementations, the system leverages distributed computing facilities, an epilepsy patient assist device. The impedance dynamics discussed herein are integrated to enhance seizure prediction and adaptive therapy.

In some implementations, the mechanisms underlying changes in extracellular space, impedance dynamics, and seizure generation are analyzed. Impedance measurements are applied to make seizure predictions.

### Example Implementation 1 Introduction

Seizures occupy a relatively small fraction of time for most people with epilepsy (PWE), their apparent unpredictability is an overwhelming challenge of living with epilepsy. Even those with drug resistant epilepsy spend less than 1% of the time experiencing a seizure, with reminder of the time spent living under the threat of a potential seizure. Currently, PWE take anti-seizure medications daily that are often short-lived and infrequent. The potential for seizure forecasting to transform epilepsy therapy and improve the lives of PWE by enabling preemptive administration of medications and electrical brain stimulation.

A comprehensive approach encompassing biomarker detection, seizure forecasting/detection, intervention, and therapeutic stimulation/medicine delivery is disclosed. In some implementations, a system that integrates tracking brain activity, seizures and behavior in a closed-loop design is disclosed, enabling efficient delivery of epilepsy healthcare to patients in their natural home environment over an extended periods.

In some alternative examples, forecasting relies on biomarkers derived from local field potential or temporal structures of seizure occurrence.

In some examples, impedance as a novel biomarker in epilepsy is used for seizure forecasting. In some implementations, information about brain impedance temporal dynamics are leveraged. For example, fast impedance changes on millisecond timescales due to the opening of ion channels in active neuron membranes, and slow impedance changes occurring over seconds to minutes attributed to cell swelling, have been observed during seizures. There is a close relationship between impedance and the brain's extracellular space (ECS), which plays a role in influencing neuronal excitability and seizures. There is a strong correlation between periodic changes in ECS, impedance oscillations, and behavioral state in the human limbic system. A significant increase in impedance level and rate of impedance change several hours before a seizure is a biomarker indicative of high likelihood of seizure occurrence. As a component of the system, impedance is periodically sampled with high temporal resolution (about minute scale) at multiple brain locations over an extended period. In some implementations, a single brain region is sampled every 5-15 minutes. By integrating the impedance dynamics information into the system, an automatic and practical system for high performance seizure forecasting and adaptive therapy is developed.

In some implementations, the systems disclosed are used for seizure forecasting and prevention and advantages include improving the quality, safety, and effectiveness of seizure forecasting and intervention. Additionally, by incorporating information about impedance dynamics into the system, performance in seizure forecasting and adaptive therapy is improved, thereby allowing for the prevention of seizures before they occur. These forecasting models could use a machine learning method including deep learning, support vector machine (SVM) or random forest (RF). Example implementation 1 results.

In the example implementation, the following subjects and data was acquired. The pilot data came from a range of animal and human investigation into the temporal dynamics of brain impedance spectroscopy. Five human subjects diagnosed with drug-resistant bilateral mesial temporal lobe epilepsy were implanted with investigational Medtronic Summit RC+S™ devices targeting the bilateral anterior nucleus of the thalamus and bilateral mesial temporal structures. Each subject was implanted with four Platinum-Iridium (Pt—Ir) alloy leads, each containing four electrodes (contacts), resulting in 16 channels per subject. The leads were implanted in the left and right thalamus (THL), amygdala-hippocampus (AMG-HPC), and posterior hippocampus (post-HPC).

Electrical two-point monopolar impedance measurements were nonuniformly sampled every 5-15 minutes and transmitted to a cloud database through a wireless network. The effective impedance (Z) was calculated by injecting a single square-wave current pulse (I, 80 μs width), sensing the corresponding voltage (V), and applying the Ohm's low Z=V/I. This is equivalent to an impedance measured by injecting a 1 kHz sinusoidal current with an amplitude of 500 nA. Both the current injection and voltage sensing were delivered using the same electrodes.

In the example implementation, a cross-correlation estimation between seizures and impedance was made. The temporal dynamics of impedance around seizures were characterized by estimating the cross-correlation between seizure onset times and the measured impedance. To eliminate the slow impedance drift over an extended period, the impedance values were normalized by subtracting the daily impedance average. For each seizure, impedance values within a ±5-minute and a ±12-hour window surrounding the seizure onset were collected. The impedance measured across anatomical locations and across the five subjects by aligning at the onsets (FIG. 3, blue curves) were aggregated. To estimate the slow impedance changes within the ±5-minute window, a one-minute window was used to smooth the impedance. This procedure was repeated 100 times using bootstrap resampling techniques to obtain the mean and error band (FIG. 3A). Similarly, for very-slow impedance change within the ±12-hour window, a one-hour window was used to obtain the mean and error band of the smoothed impedance surrounding the seizure onsets (FIG. 3B).

The significance of the observed impedance variation was assessed by calculating the baseline signal of impedance change. Time-matched interictal (days w/o seizures) surrogate data timestamps was generated according to the distribution of seizure onsets and repeated the same estimation procedure used for days with seizures. The baseline interictal data is from days without any seizures within ±12 hours (FIG. 3, green curves).

In the example implementation, slow and very-slow seizure-related impedance changes were analyzed. Both the level and rate of change in impedance surrounding seizure onsets (FIG. 3) were analyzed. The results show significant elevation of slow impedance changes (more than 6 SEM) around the onsets in the hippocampus region within a ±5-minute window (FIG. 3A, top row: AMG-HPC and Post. HPC structures). Impedance increases prior to unprovoked spontaneously occurring seizures in humans. In some examples, a different pattern was observed in THL. For example, the rate of impedance change showed significant increases before seizure onset in all three areas (FIG. 3A, bottom row), approximately 2-4 minutes prior to onset in THL, 1-3 minutes in AMG-HPC and Post. HPC.

The results of very-slow impedance changes also suggest an increase in impedance level around seizure onsets (about 1 hour before and 4 hours after) within a ±12-hour window in hippocampus area (FIG. 3B, top row). All three anatomical locations indicate a significant increase in impedance change before seizure onset. However, the increase is more pronounced in the hippocampus region, occurring about 1-3 hours prior to the onset (FIG. 3B, bottom row). This significance of the cross-correlation, particularly 1-3 hours before seizure onset, forms a basis of the proposed forecasting methodology.

In some implementations, an integrated seizure forecasting system with closed-loop design is disclosed. In some implementations, a seizure forecasting system utilizes impedance spectroscopy dynamics.

FIG. 4 illustrates a schematic diagram of the seizure forecasting system based on impedance dynamics.

The implanted electrodes (A) are surgically implanted in various locations within the brain's limbic system. In some implementations, the implanted electrodes (A) are Pt—Ir electrodes. The brain's impedance is periodically sampled by injecting current into the brain tissue and sensing the corresponding voltage signals.

The implanted device (b) device manages the measurement of impedance among other signals and can optionally initiate preventive brain stimulation. In some examples, the system is agnostic to any implantable sensing and stimulation device with bi-directional wireless streaming for off-the-body analytics.

The electronic personal assistance device (EPAD) (C) is a handheld device (e.g., running on smartphone) that is configured to perform multiple functions, including relaying data from the implanted device to the cloud, broadcasting visual/audio alarms upon receiving a seizure risk alert, and receiving patient inputs. In some implementations, the EPAD (C) has sufficient edge computing capacity, and can run the deep-learning (DL) forecasting model directly.

The cloud co-processor (d) trains the deep learning (DL) model. The probability of the predicted seizure risk can also be computed in the cloud when the EPAD (C) does not have adequate computing bandwidth. In some implementations, an adaptation of Bi-directional Long Short-Term Memory (BiLS™) model for seizure forecasting is used. The original impedance data is segmented, and additional inputs such as the rate of impedance change, and time-frequency power signals of each segment are added to the model input. The input is then fed into a parallel 1-D convolution network (CNN) for feature extraction. The subsequent full connection layer constructs feature representation, which goes to the next BiLS™ layer to learn valid information from the noisy data through forward/backward propagation. An attention mechanism is then employed to redistribute the weights and emphasize the critical features of preictal stage from a long sequence of signals. Finally, a full connection layer outputs the preictal probability. The specific threshold for classification will be determined by forecasting performance requirements.

The monitoring station (e) receives estimated seizure risk and other related information from the cloud co-processor (d) in real-time (or near-real time), providing critical patient information. In some implementations, the monitoring station (e) is located at a designated medical center, where the station receives estimated seizure risk and other related information from the cloud co-processor (d) in real-time, providing critical patient information to the medical/research team. In some implementations, the monitoring station (e) may automatically send seizure alarm signals to EPAD (C).

In some implementations, a Medical/Research Team (F) reviews the automatic seizure alert signals and makes the final decision regarding sending warning signals to patients.

FIGS. 3A and 3B present analysis of impedance dynamics related to seizures. FIG. 3A presents the impedance levels and rate of change within a ±5-min window surrounding seizure onset (indicated by red vertical lines), compiled from five subjects. The top row illustrates the impedance level associated with actual seizures (blue) and baseline events (green, time-matched seizure-free epochs) in the three anatomical locations under investigation: the Thalamus (THL), Amygdala-Hippocampus (AMG-HPC), and Posterior Hippocampus (Post. HPC). Shaded regions denote significant difference between the actual and baseline impedance levels, with lower-half shading indicating lower levels than baseline epoch impedance levels, and upper-half shading indicating higher impedance levels. The bottom row depicts the rate of impedance change associated with actual seizures (blue) and baseline time-matched seizure free epochs (green). FIG. 3B shows the impedance levels and rate of change within a ±12-hours window surrounding seizure onset. The legends are consistent with FIG. 3A. In both FIGS. 3A and 3B, blue and green error bands indicate ±3 SEM, where the following abbreviations are used: Norm., Normalized; imp., impedance; THL, thalamus; AMG-HPC, Amygdala-Hippocampus; Post. HPC, Posterior Hippocampus.

FIG. 4 illustrates a schematic diagram of the seizure forecasting system based on impedance dynamics. The system operates in a closed-loop manner, with impedance periodically sampled via (A) implanted electrodes at multiple brain locations over an extended period. The measurement process is managed by the (B) implanted device (e.g., Medtronic RC+S™), which wirelessly transmits the measurements to the patient's (C) Electronic Personal Assistance Device (EPAD). The EPAD then streams the data to the (D) cloud co-processor via the internet, where a deep-learning model (described herein) computes the probability of predicted seizure risk at each time instance. The estimated seizure risk and other related information are sent back to (E) the monitoring station at a designated medical center, where (F) a medical/research team can check critical patient information in real-time. Seizure risk information can be automatically fed back to (C) the patient's handheld device, or a physician can quickly review the automatic estimation and decide whether to send an alarm to the patient. Upon receiving the seizure risk warning, the EPAD will broadcast audio and visual alarms to the patient. Optionally, the EPAD can send signals to the (B) implanted device to initiate preventive deep brain or cortical therapeutic stimulation through the (A) implanted electrodes. The integrated system provides comprehensive seizure monitoring, forecasting, and adaptive neuromodulation therapy with high performance. The following abbreviation is used in FIG. 4: Thres., threshold.

### Example Implementation 2

Impedance is an electrical property of brain tissue and plays role in shaping the characteristics of local field potentials, the extent of ephaptic coupling, and the volume of tissue activated by externally applied electrical brain stimulation. Brain impedance, sleep-wake behavioral state, and epileptiform activity were tracked in five people with epilepsy living in their natural environment using an investigational device. The example implementation identified impedance oscillations that span hours to weeks in the amygdala, hippocampus, and anterior nucleus thalamus. The impedance in these limbic brain regions exhibit multiscale cycles with ultradian (˜1.5-1.7 h), circadian (˜21.6-26.4 h), and infradian (˜20-33 d) periods. The ultradian and circadian period cycles are driven by sleep-wake state transitions between wakefulness, nonrapid eye movement (NREM) sleep, and rapid eye movement (REM) sleep. Limbic brain tissue impedance reaches a minimum value in NREM sleep, intermediate values in REM sleep, and rises through the day during wakefulness, reaching a maximum in the early evening before sleep onset. Infradian (˜20-33 d) impedance cycles were not associated with a distinct behavioral correlate. Brain tissue impedance is known to strongly depend on the extracellular space (ECS) volume, and the findings reported here are consistent with sleep-wake-dependent ECS volume changes recently observed in the rodent cortex related to the brain glymphatic system. In this example implementation, human limbic brain ECS changes during sleep-wake state transitions underlie the observed multiscale impedance cycles. Impedance is an electrophysiological biomarker that this implementation found useful for tracking ECS dynamics in human health, disease, and therapy.

### Example Implementation 2 Introduction

Electrical impedance is an extrinsic tissue property that characterizes the frequency-dependent resistance to endogenously generated ionic currents and externally applied electrical stimulation currents in the brain. The impedance influences the spatiotemporal dynamics of extracellular ionic currents that give rise to local field potentials (LFPs) and determines the range of ephaptic coupling and the volume of tissue activated by electrical brain stimulation (EBS). The voltage generated in response to an externally applied current can be used to probe and track brain impedance, and at the scale of clinical EBS electrodes (˜mm²), the impedance depends on the electrode-tissue interface and ionic current conductance through the intracellular, vascular, and extracellular space (ECS) brain compartments. The impedance of rodent, nonhuman primate, and human brain tissue is primarily resistive over the frequency range of physiologic LFP and is strongly dependent on the ECS volume fraction. The interstitial conductive fluid filled ECS creates a complex electrical network of ionic current flow that is increasingly recognized for its role in sleep-dependent brain health, such as clearing neurotoxic metabolites and pathologic proteins associated with neurodegenerative disorders.

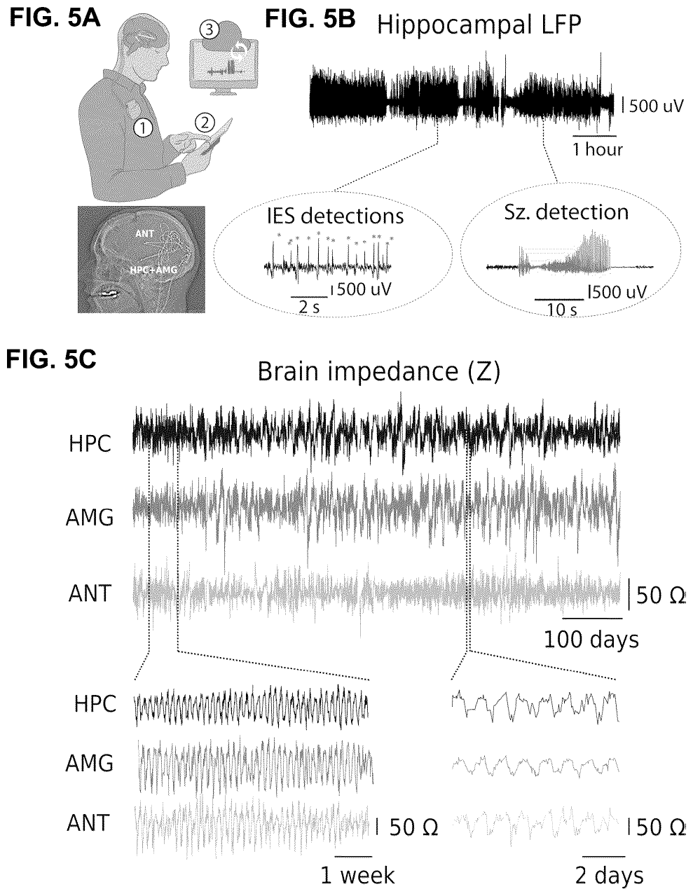

In this example implementation, it was hypothesized that if sleep-wake transitions drive changes in human brain ECS volume, and manifest as impedance cycles with lower impedance in NREM sleep, given the increased ECS volume compared with wakefulness and REM sleep. In this example implementation, an investigational implantable neural sensing and stimulation device was used to continuously track impedance, sleep-wake behavioral state, and epileptiform activity in anterior nucleus of thalamus (ANT), amygdala (AMG), and hippocampus (HPC) over several months in five people with drug-resistant mesial temporal lobe epilepsy living in their natural home environment (FIG. 5).

### Example Implementation 2 Materials and Methods

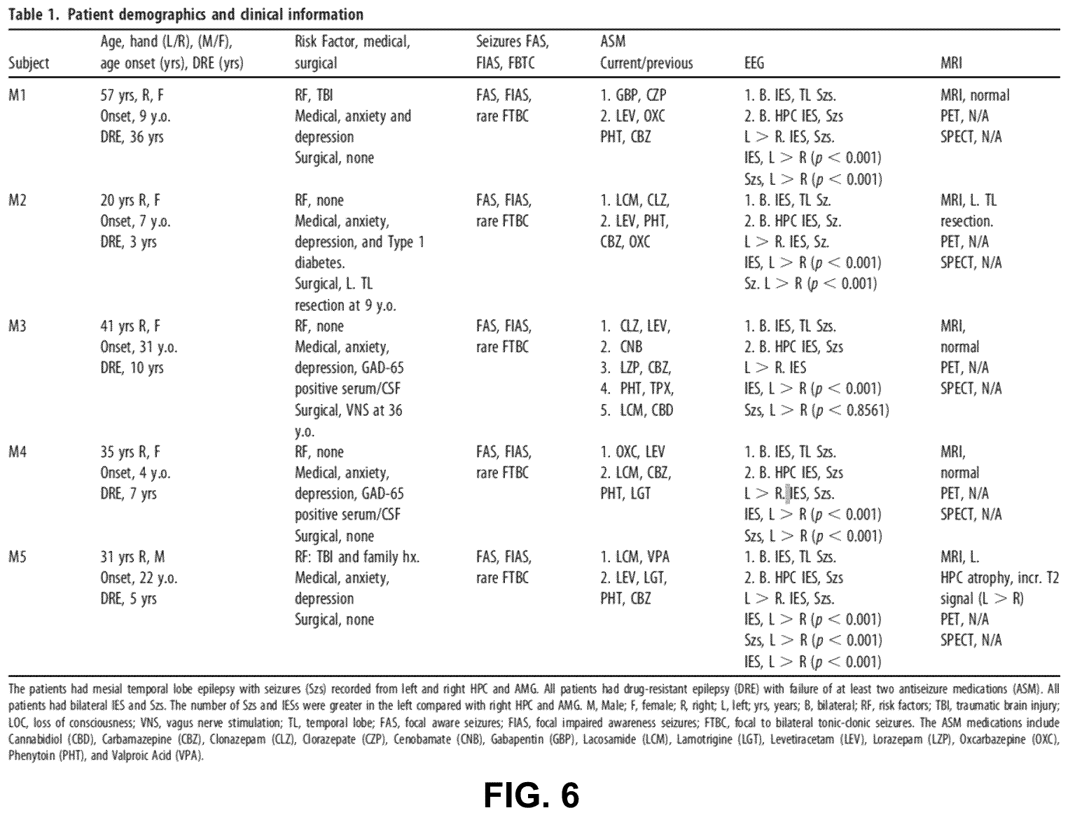

This example implementation people with drug-resistant mesial temporal lobe epilepsy were investigated. Seven subjects with drug resistant mesial temporal lobe epilepsy were identified for the study. Five subjects ultimately met the full inclusion criteria with adequate numbers of baseline seizures and were implanted with the investigational neural sensing and stimulation device. The demographics and clinical information of the five subjects implanted with the Medtronic RC+S are provided in the table shown in FIG. 6.

The epilepsy patient assistant (EPA) is a custom software application running on a handheld computer enabling bidirectional communication between the implanted device, wearables, and cloud computing resources. The EPA features include automated algorithms for continuous LFP data acquisition, electrical stimulation, impedance testing, LFP analysis and an interface for collecting patient interactions.

The investigational Medtronic RC+S is an investigational implantable device with bidirectional wireless communication capability, programmable 16-channel electrical stimulation, two-point impedance measurements, and continuous four-channel (selected bipolar pairs) wireless LFP streaming to a handheld computer and cloud environment

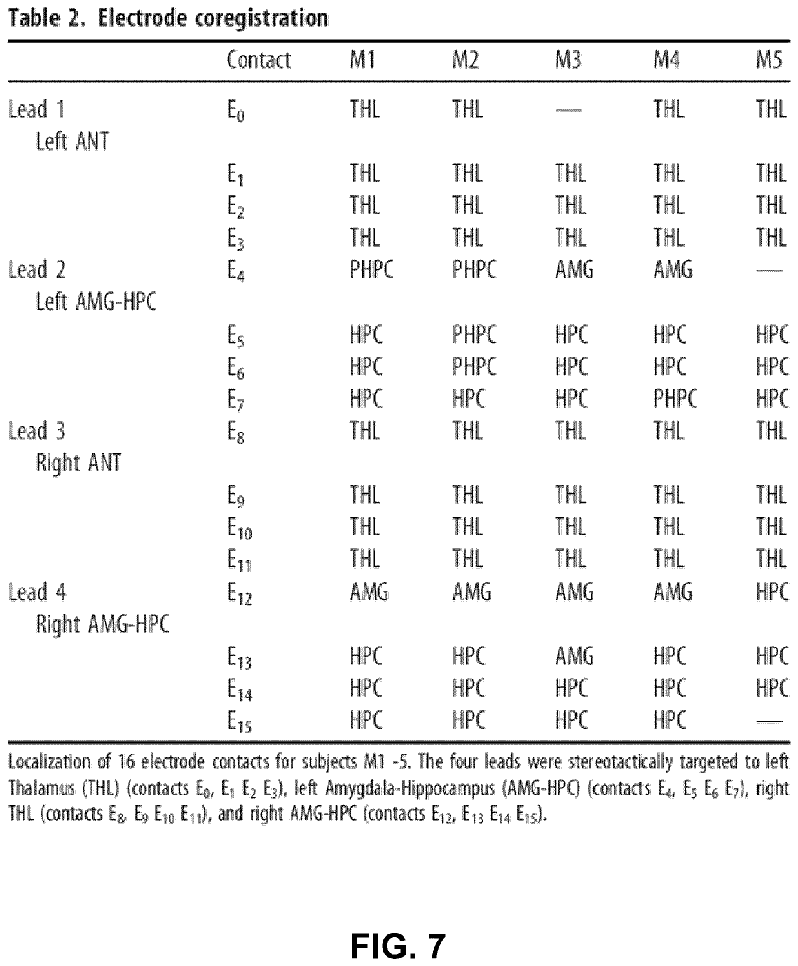

For lead and electrode contact localization (FIG. 5A), five subjects (M1, 2, 3, 4, 5) had four leads stereotactically implanted into bilateral ANT and bilateral AMG-HPC. All five subjects had Medtronic 3387 implanted in the ANT. All subjects had AMG-HPC sites implanted with Medtronic 3391 leads, except the left HPC of M2. Subject M2 had previously undergone a left anterior temporal lobectomy leaving only a residual left HPC tail that was implanted with a Medtronic 3387-lead. Subject M5 does not have a right AMG electrode contact because of a technical problem at the time of surgery. The 3391-lead tail could not be fully seated into the lead extension connector, leaving only three of the four contacts recording. For each patient, the four leads (16 total electrode contacts) implanted were localized with postoperative CT scan coregistered to the preoperative MRI for anatomic localization. The CT scan and electrode contact positions were coregistered to a T1-weighted anatomic MRI scan using coregistration in SPM12 software. FreeSurfer software was used to segment the T1-weighted MRI and the electrodes labeled according to the Destrieux atlas. The final electrode contact localization for impedance analysis was performed with the Lead DBS toolbox. FIG. 7 provides the localization information for the electrode contacts used in the study.

In some implementations, the LFP is sensed using the RC+S device. The LFPs from left and right ANT, AMG, and HPC using four bipolar electrode pairs, sampled at a frequency of 250-500 Hz, were selected for continuous data streaming. The contacts used to create lead-specific bipolar recordings were selected by visual review of LFP data during seizures, resting wakefulness, and sleep-wake transitions. In some implementations, the ANT electrode contacts were not used for LFP recordings or further analysis.

The sensed LFP is analyzed. The LFP analysis was performed retrospectively. Previously validated automated algorithms were applied to the long-term intracranial LFP recordings to identify seizures, IES and classify wake-sleep state (Awake, REM, and NREM). The algorithm pipeline identifies seizures, IES and sleep-wake behavioral state for consecutive 30 s data segments. The sleep-wake classifications and seizure events were then synchronized with the impedance measurements for further analysis. Toolboxes and data for automated LFP analysis for detecting IES, seizures, and performing sleep-wake classification were developed and used.

Seizures and IES are detected from continuous LFP recordings. Interictal epileptiform spikes and seizures are electrographic biomarkers of pathologic, epileptogenic brain tissue and are readily identified by human visual review of LFP recordings. Continuous hippocampal LFP recordings from the five subjects were visually reviewed and IES and seizures labeled for subsequent use in training, validating, and testing automated detectors.

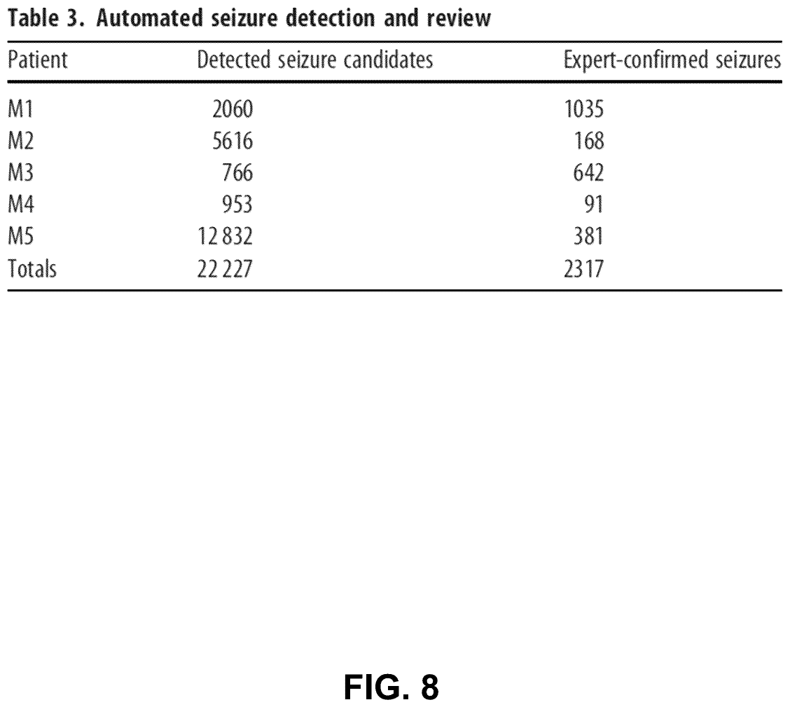

A validated algorithm for detecting IES transients from LFP recordings was used. The adaptive IES algorithm enables detecting IES in long-term LFP data with changing background activity commonly encountered in prolonged recordings spanning weeks of time. The training data was used to set a hypersensitive threshold for all subjects (FIG. 8).

The intracranial LFP associated with epileptic seizures exhibits characteristic temporal and spectral evolution over a wide frequency range (FIG. 6). An accurate seizure detector was developed using a convolutional neural network (CNN) with long-short-term memory (LSTM) using short-time Fourier transform of the LFP as the input. The CNN-LSTM outputs a seizure probability for 10 s data segments. Gold standard visually reviewed seizures are used for training, validation, and pseudoprospective testing. The seizure detection performance using the CNN-LSTM model is applied using a hypersensitive threshold to ensure all seizures are detected for the impedance analysis. All candidate seizure events detected by the hypersensitive automated CNN-LSTM were visually reviewed (FIG. 8).

The temporal distribution of seizures was determined by plotting the circular histogram of seizure onset times for all verified seizures across all subjects. To compare the impedance profiles during seizures with those observed outside seizure events, surrogate nonseizure data were generated. These surrogate nonseizure data were carefully aligned to match the time-of-day distribution observed for actual seizures. The generation of surrogate nonseizure data involved simulating 24 h periods without any seizure activity and ensuring equivalent distributions of times for impedance comparison. Importantly, the surrogate data account for the natural circadian impedance cycles. To create the estimated probability distribution of seizures, a rejection method was used based on the geometric interpretation of probability distribution. This method allowed for modeling the probability distribution of seizure occurrences and evaluate the impedance patterns specific to seizure events against the baseline nonseizure impedance data.

In some examples, sleep-wake classification is made from continuous LFP recordings. Reliable sleep-wake classification was determined by training a subject-specific classifier on simultaneous LFP and gold standard sleep annotations from polysomnography (PSG). For each subject over the course of three nights in the hospital epilepsy monitoring unit, we recorded simultaneous scalp PSG and continuous intracranial LFP data. The PSG signals were scored into standard sleep categories (Awake and REM) and two NREM stages (N2, N3) using American Academy of Sleep Medicine 2012 scoring rules. A subject-specific sleep classifier (Naïve Bayes) was trained using the first night of data. The second night was used for validation and the third night for pseudoprospective testing. The Naïve Bayes classifier uses features extracted from the LFP that were determined to be useful for sleep-wake behavioral state classification.

Electrical impedance is measured and analyzed. The two-point monopolar electrical impedance was measured from all electrodes in AMG, HPC, and ANT using a square-wave current pulse (0.4 mA, 80 μs pulse width) over multiple months in five people with drug-resistant epilepsy. In some implementations, the AMG-HPC electrode contacts in seizure onset tissue and ANT electrode contacts used for therapeutic EBS were excluded from impedance analysis.

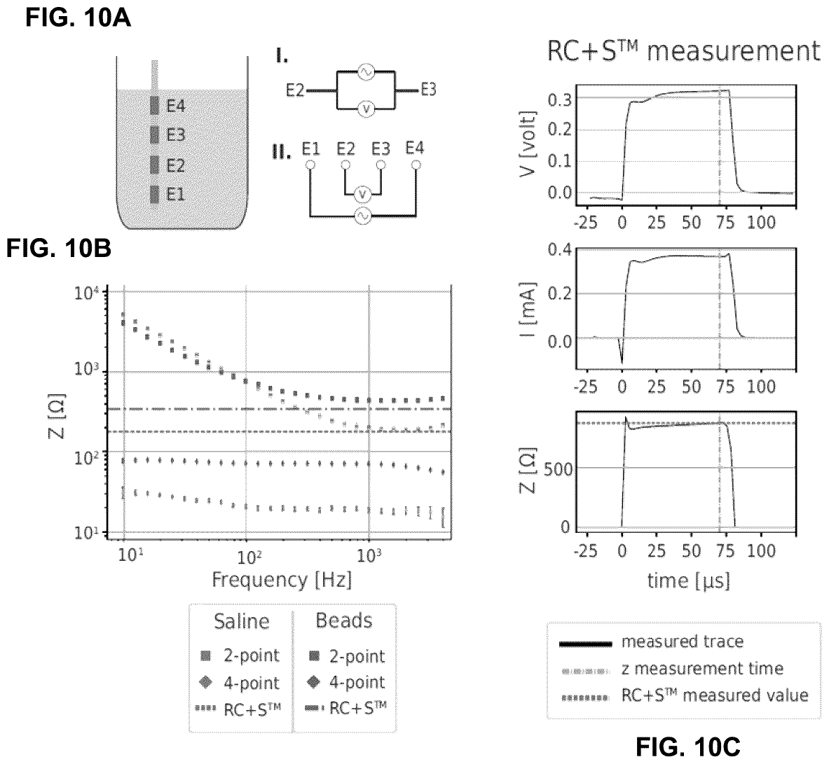

The RC+S two-electrode impedance method uses the same electrode contacts for delivering current stimulation and sensing the voltage response. The voltage response to the applied current pulse using the two-point method includes the electrode-tissue interface polarization, created by the electrical stimulation, in addition to the bulk tissue impedance. To address the electrode tissue interface contribution caused by electrolyte polarization, the four impedance calculation can be used. This method involves using one pair of electrodes to deliver the current and a separate pair of electrodes to measure the voltage response (FIG. 10). In some implementations, the impedance sensing device supports two-point impedance measurements. In some implementations, the impedance sensing device supports four-point impedance measurements. To investigate and compare the differences between two-point and four-point measurements, a series of benchtop experiments were conducted. These experiments involved using a composite material consisting of saline and microbeads.

A composite model was used to compare the impedance measurements obtained with the two-point square-wave pulse current probe the RC+S device uses with the results obtained from the four-electrode and two-electrode measurement using sinusoidal stimulation currents.

In some implementations, saline/microbead impedance is measured. Benchtop experiments were conducted using saline and saline/microbead composites (FIG. 10). The aim of these experiments was to directly compare two-point and four-point electrode impedance measurements over a range of sinusoidal current frequency (1-5000 Hz) with two-point RC+S impedance measurements using a single square-wave pulse. A Medtronic 3387-lead (four contacts each with 4.7 mm² surface area) submerged in the center of a plastic cylinder reservoir (radius, 5 cm; height, 40 cm) filled with saline (0.9% sodium chloride solution at 22° C.; 68.9 Ω-cm), or a saline/microbead composite (volume fractions 36% saline and 64% microbeads (80-120 μm)) was used for the experiments. To simulate monopolar electrical impedance measurements, a single electrode, contact on the 3387-lead, was used as the cathode, and a large surface area electrode at the bottom of the cylinder was the anode (simulating the large RC+S surface area of ˜5.4×10³ mm²). In addition, two-point bipolar measurements using the inner two contacts of the 3387-lead were used for stimulation and recording. The four-point measurements used the outer two contacts of the 3387-lead for injecting current and the inner two contacts for recording the voltage response. A National Instruments USB6251 signal generator and a NPI electronics ISO isolation unit were used to deliver 0.4 mA sinusoidal currents over a wide frequency range (1-5000 Hz). The voltage drop across the measurement electrodes was recorded using a Neuralynx cube acquisition system. The impedance for each frequency (f) was calculated from Ohms law Z_{eff} (f)=V_{measured} (f)/Iₛₜᵢₘ (f) and averaging the measurement at each frequency over >25 cycles.

The two-point RC+S impedance measurement uses a single current pulse (0.4 mA, 80 μs pulse-width) to probe the tissue and calculates the impedance from Ohm's law Z_{eff}=V_{measured}/Iₛₜᵢₘ with the voltage measured at 70 μs, near the end of the 80 μs current test pulse (FIG. 10C). The two-point RC+S can be directly compared with the two- and four-point impedance measurements that use sinusoidal currents (FIG. 10B).

The measured human brain impedance was analyzed. The impedance time series (sampled every 5-15 min) in the five subjects were resampled with a 30-min-long moving average window with a step of 10 min and bandpass filtered between 30 min to 100 d with low- and highpass finite impulse response, zero phase shift filters of 1,001st order.

Polar impedance plots with the histograms of the minimum and maximum impedance value recorded over each 24 h (day/night) cycle were tested for nonuniform circular distributions using the Kuiper (1960) test (*p, 0.05, **p, 0.01, ***p, 0.001).

The multiscale impedance cycle was analyzed. The continuous wavelet transform (CWT) was used to investigate brain impedance cycles with ultradian (<24 h), circadian (˜24 h), and infradian (>24 h) periodicities. The CWTs were implemented in MATLAB software (Morlet wavelets and L2 normalization). The Thomson F test multitaper scheme was used to test for significance of oscillations with a 5-day moving window used for ultradian and circadian cycles and a 100-day moving window (step of 5 days) for infradian cycles. The presence of ultradian, circadian, and infradian frequency band oscillations were identified using preprocessed impedance signals. The periodicity test in ultradian band for each 5 d signal segment was investigated using the time-half-band product (TW) set at three (TW=3), and the number of tapers (K) was five (K=5), resulting in the frequency resolution of 2 W=1.2 (or W=0.6) cycles/day. In ultradian band, frequency of interest (FOI) was chosen from 23/24=0.96 h/cycle to 6 h/cycle with an incremental step of 15 min; in infradian band, the FOI was from 2.5 d/cycle to 30 d/cycle with an incremental step of 15 min. At each fixed frequency, the F test was set at the level of the upper 99% (p=0.01) percentage point of the F distribution with 2 and 2 K−2=8 degrees of freedom under the null hypothesis of no spectral line, leading to the critical value of 8.65.

In some examples, the sleep-wake state dependence of impedance was analyzed. The three behavioral categories on a group and subject level were compared using a statistical two-tailed Mann-Whitney test (p<0.05, Bonferroni correction for multiple observations). The continuous behavioral state classifications were also reviewed for periods of daytime sleep (>30 min duration), and in four of five subjects' daytime naps were identified to analyze and comparison of sleep impedance during daytime naps and overnight sleep.

The LFP and impedance data was summarized. The RC+S provides streaming of four LFP channels (250-500 Hz sampling) and 16 monopolar impedance values (0.001-0.003 Hz sampling) to the patient's tablet computer for storage. When there is either cellular or wireless connectivity available the data are transferred from patient's tablet to a cloud database. The sampling frequencies were determined as a compromise between data quality, wireless data transmission, and device battery limitations. The LFP and impedance data were collected during an ongoing investigational device clinical trial that is testing different ANT EBS paradigms for drug-resistant epilepsy that include (1) no EBS baseline, (2) low-frequency EBS (2 and 7 Hz continuous), and (3) high-frequency EBS (125 Hz continuous, duty cycle, 1 min on and 5 min off, and responsive).

In one example, because of the acute effects on tissue impedance, the following was excluded (1) data from the initial 14 d after surgical implant, (2) data within 12 h before and after seizures, (3) data from electrode contacts located in seizure onset zone, (4) data from electrode contacts used for EBS, and (5) data from electrode contacts with high impedance (0.5000 V). This left 45.00% (36/80) of the electrode contacts (5 AMG, 14 HPC, 17 ANT) for subsequent analysis of impedance and association with sleep-wake behavioral state.

The complete dataset available for analysis included at least 150 d for each patient (M1, 828 d; M2, 549 d; M3, 346; M4, 549; M5, 150 d). In some examples, days with poor telemetry and, 70% data were excluded because of the possibility of missing seizures, leaving 1456 continuous 24 h epochs from 5 subjects (291±198 24 h epochs). The analysis of sleep-wake impedance changes was focused on the 30 d period before starting EBS. During the initial period without EBS there were 299 data segments (30-90 min durations) from the 36 electrode contacts without seizure activity for at least 24 h annotated across the five patients and three behavioral categories—Awake, NREM, REM (M1, 30/21/22; M2, 20/19/18; M3, 15/14/13; M4, 22/27/29; M5, 12/19/18) for analysis.

### Example Implementation 2 Results

The LFP recordings from the five subjects were used as input to automated algorithms for detecting seizures and classifying sleep-wake state (FIGS. 7 and 8). The characteristics of sleep show a wide range of features including sleep onset, offset and duration. The number of NREM-REM cycles over the dataset analyzed shows a significant variability within patients and across the group (FIG. 10). All five subjects had independent bilateral mesial temporal lobe IES and seizures. The IES rate and seizure counts are significantly higher in the left compared with right mesial temporal lobe (p<0.001; FIG. 6).

Saline/microbead composites were used to compare two-point RC+S impedance measurements that use a square-wave current pulse with both two-point and four-point impedance measurements using sinusoidal currents (FIG. 10). The microbead volume fraction was adjusted to approximate the impedance measured in mammalian and human brain using two-point measurements. The qualitative difference between two-point and four-point impedance measurements is the absence of frequency dispersion in the impedance over the range of ˜1-1000 Hz with the four-point measurement. The two-point measurement shows frequency dispersion over this range consistent with a capacitive term because of the Helmholtz double layer at the stimulation electrode-electrolyte interface. Above ˜2000 Hz the two-point impedance is purely resistive without dispersion. The two-point RC+S voltage response to the 0.4 mA, 80 μs square-wave probe shows behavior consistent with charging a capacitive interface over the initial ˜40 μs and then asymptotes before the measured response at 70 μs and yields an impedance similar to the impedance measured with sinusoidal currents between 1000 and 4000 Hz (FIG. 10B, dashed line). In this frequency range the two-point measurement is also purely resistive without frequency dispersion. The difference between four- and two-point measurements highlight the significant confound introduced by the electrode-media interface impedance that is in series with the brain tissue impedance when the electrical current stimulation and voltage sensing are performed with the same electrodes.

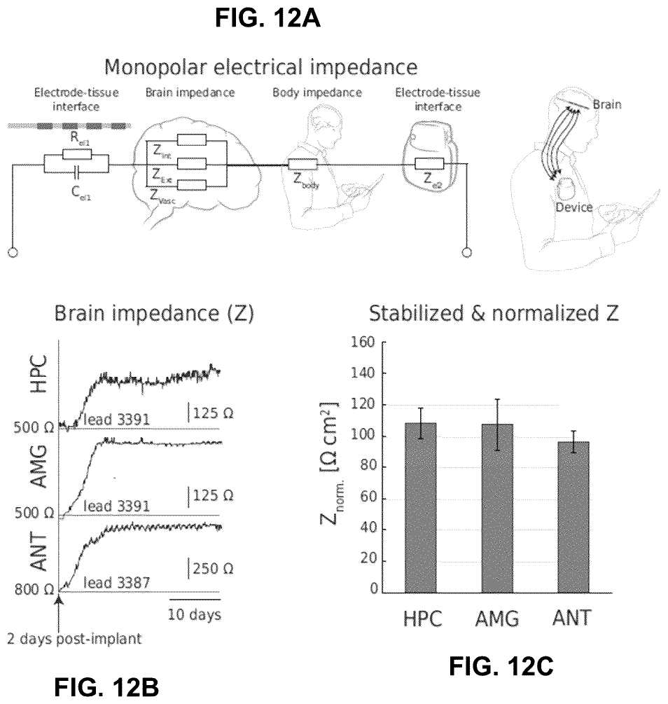

Impedance (e.g., human brain impedance) was measured in five people with drug-resistant mesial temporal lobe epilepsy over multiple months using the RC+S monopolar two-point current pulse method with periodic sampling (every 5-15 min) in AMG, ANT, and HPC. The impedance increases after surgery but reaches a stable value after ˜14 d (FIG. 12B). All subsequent analysis was performed using data at least 15 d after implant to avoid the acute effects.

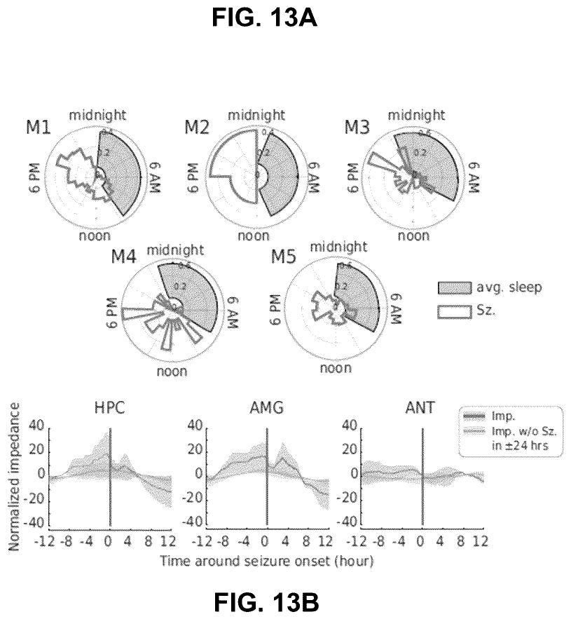

Seizures effect brain impedance and ECS volume, and to address this possible confound we used a hypersensitive seizure detector combined with expert visual review to label all seizures in the LFP data. The seizures primarily occur in either the morning or late afternoon in all subjects M1-5 (FIG. 13A). The impedance in the 24 h surrounding seizures was increased when compared with days without seizures (FIG. 13B). To eliminate the confound of seizure-related impedance changes, the ±12 h data segments around seizures are excluded from all subsequent analysis.

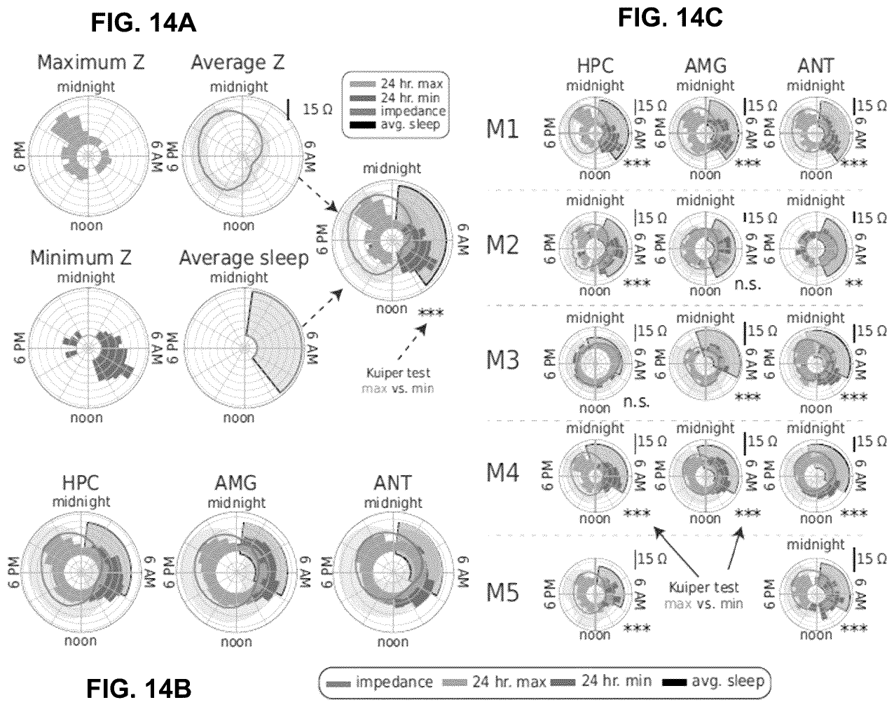

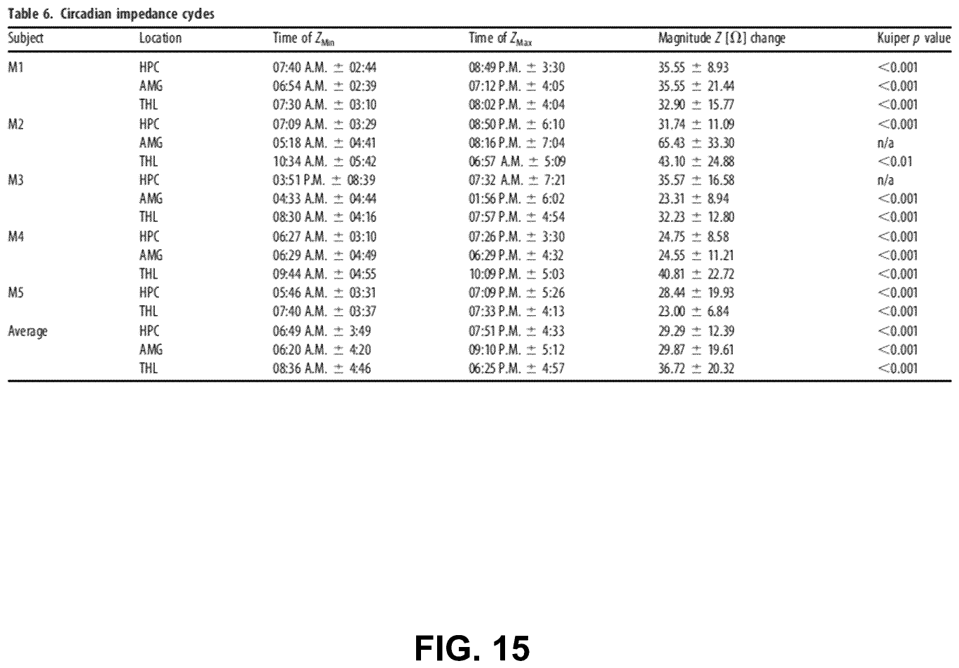

Group-level analysis (FIG. 14B) reveals that the average impedance increases throughout the day in HPC, AMG, and ANT. The average impedance peaks at 7:51 P.M.±04:33, 9:10 P.M.±(05:12), and 6:25 P.M.±(04:57) in the evening for HPC, AMG, and ANT respectively. The average impedance decreases during the night, reaching its lowest point at 6:49 A.M.±(03:49), 6:20 A.M.±(04:20), and 8:36 A.M.±(04:46). The average impedance difference between the daytime maximum and nighttime minimum was 29.29±12.39Ω for HPC, 29.87±19.61Ω for AMG, and 36.72±20.32Ω for ANT (FIG. 15). FIG. 14 shows the group-level and individual subject circular histograms of the maximum impedance (orange) and minimum impedance (brown).

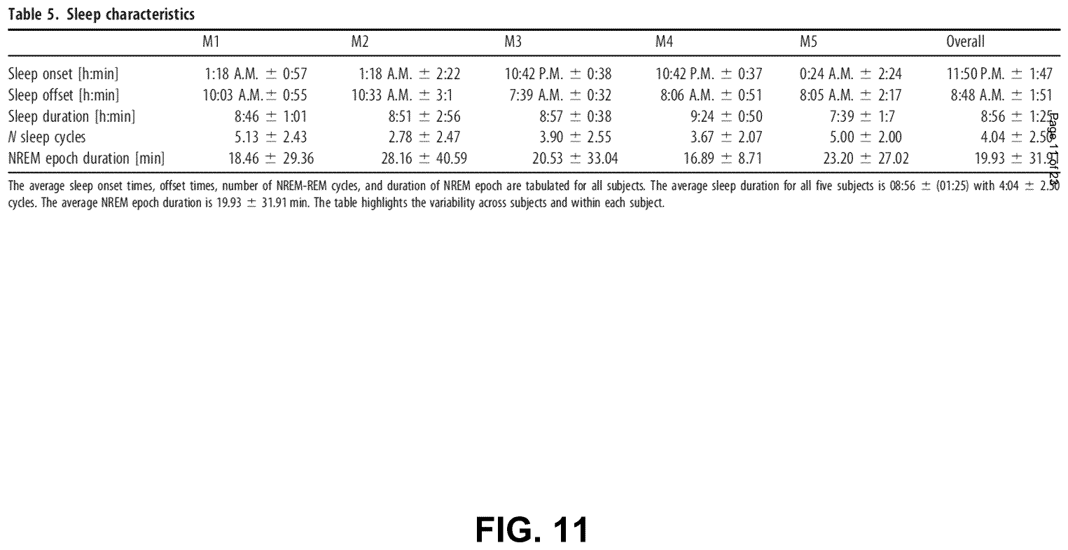

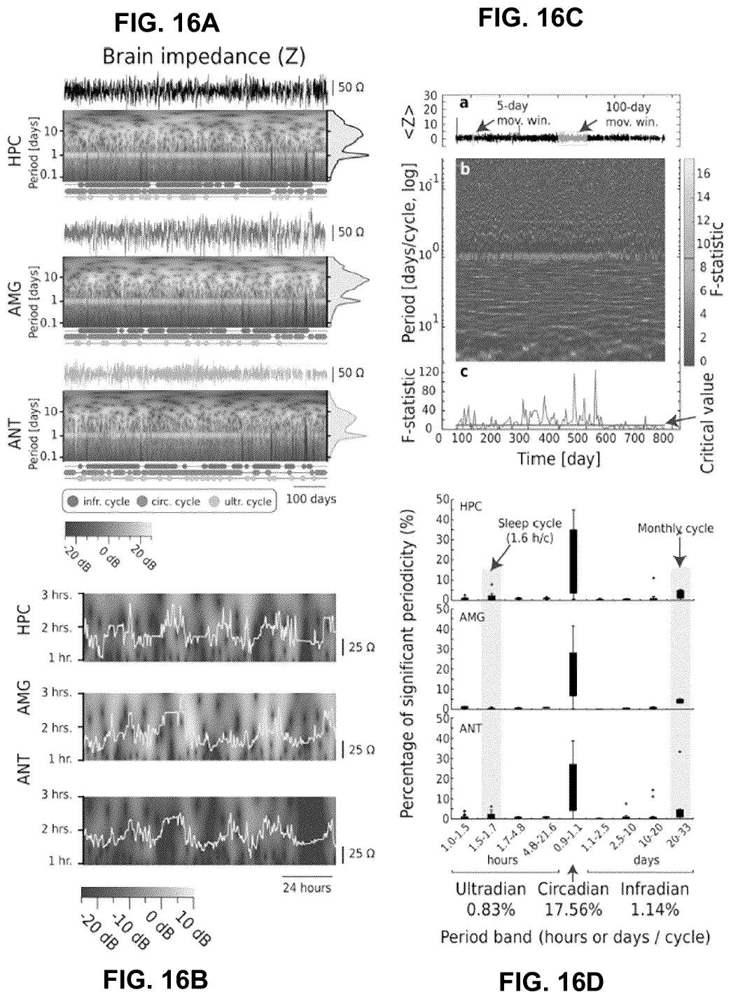

In some implementations, multiscale impedance oscillations are analyzed. In addition to ˜24 h cycles captured with the polar plots (FIG. 14) there are shorter- and longer-cycle periods in the impedance time series. Analysis using CWT of AMG, ANT, and HPC impedance time series shows multiscale oscillations with ultradian, circadian, and infradian periodicities (FIG. 16). The circadian oscillation is highly regular over the duration of the entire recording (FIG. 16A). Statistical testing with the Thomson F test multitaper scheme was used to identify ultradian, circadian, and infradian oscillations in the impedance time series [FIG. 16A, bottom, detection of significant ultradian (yellow dots), circadian (red dots), and infradian (blue dots) oscillations]. Ultradian cycles are not well visualized in the average amplitude index power density calculated from the long impedance time series (FIG. 16A, middle right, amplitude index in gray), but when investigated in shorter data segments the ultradian cycles are more readily visualized (FIG. 16B). The variability of sleep onset and offset times and sleep duration (FIG. 11) all contribute to the reduced average amplitude index power density. When the oscillation detections are aggregated, the cycle periodicities occurring most frequently can be identified (FIG. 16D). Note that in the ultradian band centered at 1.5-1.7 h/cycle is increased, which is the average human NREM-REM sleep cycle (1.6 h/cycle, or 5 cycles per 8 h).

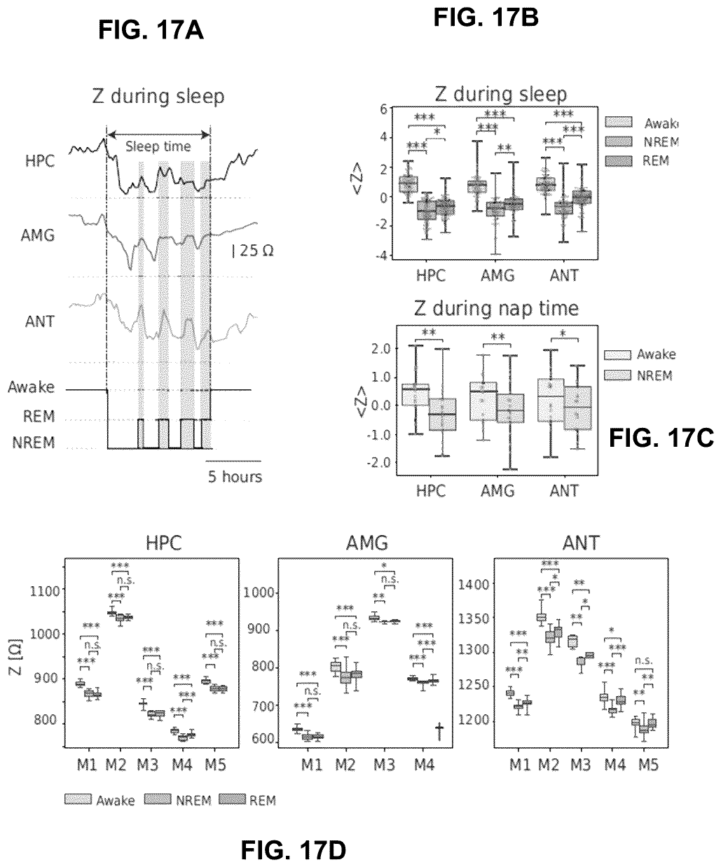

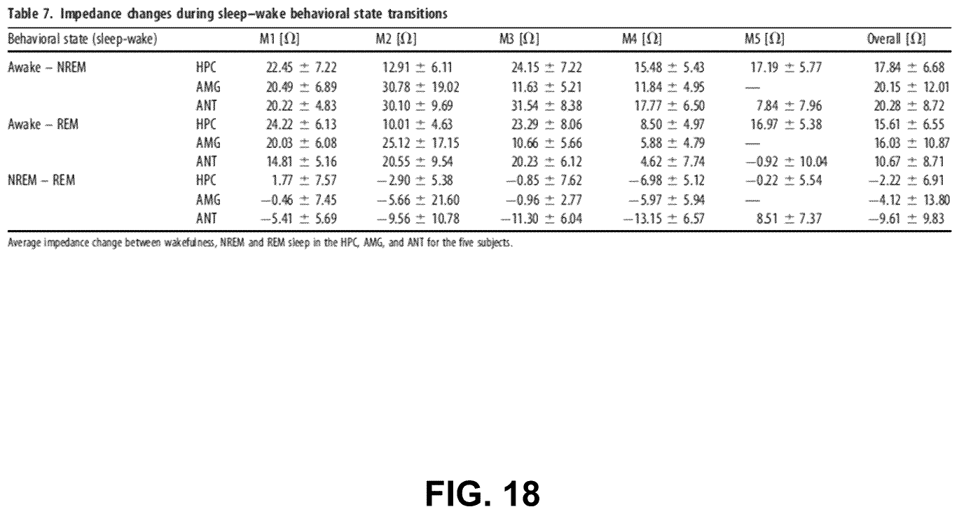

In some implementations, correlations between impedance and behavioral states (wakefulness, NREM, and REM sleep) were analyzed. In total, 299 data segments from the 36 right-hemisphere electrode contacts without seizure activity for at least 24 h were identified across the five patients and three behavioral states. The impedance was lower in NREM sleep compared with wakefulness and REM sleep (FIG. 17; FIG. 18). The impedance in AMG, ANT, and HPC reaches its minimum value in NREM, intermediate value in REM, and highest value in wakefulness (FIGS. 17B and 17C).

Four of the five subjects also took afternoon naps (nap onset 2:06 P.M.±1.2 h). Impedance in NREM sleep during naps was analyzed (M1, 7; M2, 1; M3, 7; M4, 3) and compared with the wakefulness impedance in the hour before the nap. The nap NREM sleep impedance is decreased when compared with wakefulness in HPC, AMG, and ANT (FIG. 17C).

The average baseline total impedance in the AMG, HPC, and ANT during wakefulness was 753.58±80.23 Ω, 892.74±97.27Ω, and 1268.28±63.17Ω, and during NREM sleep was reduced to 730.11±77.00 Ω, 874.30±99.49Ω, and 1246.61±54.35Ω, respectively (FIG. 17D). The average circadian minimum to maximum impedance changes, DZ, in AMG, HPC, and ANT are 29.87±19.61 Ω, 29.29±12.39Ω, and 36.72±20.32Ω, respectively (FIG. 15).

### Example Implementation 2 Discussion

The total impedance consists of the electrode-tissue impedance in series with the brain tissue impedance (FIG. 12), and with the two-point measurements the electrode-tissue impedance dominates the total impedance. This is evident in the benchtop experiments (FIG. 10) in both saline and saline/microbead composites where the four-point impedance measurement is at least an order of magnitude smaller than the two-point measurement. Similarly, based on the benchtop saline/microbead experiments, we expect the human brain electrode-tissue impedance is >10 times greater than the isolated brain impedance (FIGS. 10B and 12B). The circadian cycle impedance change typically represents <5% of the total impedance. But despite the small relative impedance change, the impedance cycles with circadian periodicity are often visible in the raw data (FIG. 12B).

The human AMG, ANT, and HPC show complex temporal dynamics with multiscale oscillations spanning hours to weeks (FIGS. 5, 14, and 16). The impedance in the limbic structures reaches the lowest value in NREM, an intermediate value in REM, and is highest values during wakefulness (FIG. 17). The impedance rises on average throughout the day during wakefulness, reaching a maximum in the evening before sleep and then decreases over the night reaching a minimum value in the early morning before awakening (FIG. 14). In sleep the impedance cycles in association with sleep state transitions between NREM and REM sleep (FIG. 16).

Sleep-wake state transitions appear to generate the observed impedance cycles with characteristic oscillations anchored by a highly stable, dominant 24 h cycle. Compared with the circadian cycles, the ultradian and infradian cycles show more variability (FIG. 16). The variability of sleep characteristics (FIG. 11), NREM-REM cycles (average 4.04±2.50 cycles), and NREM epoch duration (19.93±31.91 min) will tend to smear out the CWT color maps for the ultradian periods. The significance testing on 5 d windows, however, continues to show cycles with ultradian periods that are consistent with NREM-REM cycles. The multiscale impedance cycles is shown to represent normal brain physiology given that this analysis is focused on the less-epileptogenic brain regions and excludes seizures. Electrodes and data segments not involved in seizure onset were selected and analyzed 24 h data periods without any seizure activity. The variability of NREM and REM sleep duration and state transitions observed over the multiple-month ambulatory recordings likely underlies the ultradian impedance cycle variability. This variability may be amplified here by sleep fragmentation in people with epilepsy.

In some examples, ECS volume changes drive the ultradian and circadian impedance cycles in human brain given the strong dependence of electrical impedance on ECS volume and recent demonstration of ECS volume expansion in rodent cortex during NREM sleep. The impedance data are indirect evidence that sleep-wake state transitions (wakefulness, NREM, and REM sleep) differentially modulate human ECS and is consistent with the ECS dynamics proposed for a human brain glymphatic system. The vascular and intracellular compartment conductances will have limited contributions to composite impedance with the current probe used here because of the high impedance of the blood-brain barrier and neuronal and glial cell membranes. The electrode-tissue interface contribution to the impedance is not expected to depend on the sleep-wake state and should not differentially contribute to impedance changes.

The interstitial-fluid-filled ECS at the physiological volume fraction (˜0.2) and complex cellular geometry provides a highly conductive network that may be near the percolation threshold enabling significant impedance changes with relatively small ECS volume changes.

It was estimated that the ECS volume change, ΔV_{ECS}, that would produce the observed circadian impedance change ΔZ. The impedance change is related to the change in effective specific brain conductivity ΔZ=1/4παΔσσ_{eff} where α is the cathode/anode stimulation electrode separation. The effective specific conductivity of a composite media of cells embedded in the ECS volume is Δσ_{eff}=(3/2) σ_{isf} ΔV_{ecs}, where σ_{isf} is the conductivity of the interstitial fluid. Usings σ_{isf}≅1:79 S/m for the conductivity of the ECS interstitial fluid and adjusting the ECS percolation threshold volume fraction to 0.07, a brain-specific conductivity of ˜0.250 S/m yields reasonable agreement with reported ECS volume fraction of ˜0.16.

In the real-time iontophoretic tetramethylammonium diffusion studies in mice the ECS volume in wakefulness was 0.136 and expanded to 0.227 in the anesthetic state, a AV_{ECS}≈0:09 which is a 67% relative volume change. In the composite conductivity model above this corresponds to a change in specific conductivity of Δσ_{eff}=0.24 S/m. A measured ΔZ≈30Ω in human AMG and HPC corresponds to a specific conductivity change of Δσ_{eff}=0.29 S/m from wakefulness to NREM sleep and a corresponding ΔV_{ECS}=0.10: Based on this model, the human ECS volume change associated with transition to NREM sleep is slightly larger than what is observed in mice.

In some examples, ECS volume dynamics underlie the observed sleep-wake state dependence and multiscale impedance cycles. Alternatively, sleep state-dependent change in astrocyte membrane impedance combined with a gap-junction-mediated astrocytic syncytium network could provide a low impedance pathway. Additionally, changes in ionic conductivity because of ion concentration changes in the interstitial fluid could have an effect. Given the previous two-photon imaging and real-time iontophoretic tetramethylammonium diffusion findings in mouse cortex during NREM and anesthesia, the observed ECS volume changes would have a large effect on impedance.

In some examples, the data segments selected were without seizures and brain electrodes with low IES rates were and not involved in seizure onset there is electrophysiological evidence that the brain regions analyzed are not entirely normal. There are IESs throughout the limbic network in all subjects (FIG. 6). The impedance fluctuations on the days with seizures, which are excluded in the example analysis outlined above, show impedance differences compared with days without seizures (FIG. 13). Seizures tend to occur at specific times of the day and the impedance is higher on the days with seizures (FIG. 13). The epileptic brain impedance may be changed at baseline and vary with seizure risk. The two-point impedance measurements introduce electrode-tissue interface effects and frequency dispersion (FIG. 10). Electrical stimulation acutely decreases baseline impedance without affecting the presence or characteristics of circadian impedance cycles further supports that impedance cycles are not related to changes at the electrode-tissue interface.

FIGS. 5A-C relate to monitoring human brain electrophysiology and behavior. Continuous wireless streaming of LFPs and brain impedance (Z) was used to investigate and track human brain electrophysiology in five ambulatory subjects with mesial temporal lobe epilepsy living in their natural home environment.

FIG. 5A illustrates an EPA system that enables synchronized bidirectional communications between patient, implantable neural sensing, stimulation devices, and local and/or distributed cloud computing infrastructure. The system consists of (1) RC+S implanted in a surgically created subclavicular pocket and connected to four electrode leads implanted in bilateral ANT and AMG-HPC. Bottom, Lateral xray of the bilateral ANT (3387-leads) and bilateral AMG-HPC (3391 lead) targets. The 3391-lead has four contacts (surface area, 11.97 mm²) spanning 24.5 mm. The contacts are 3.0 mm long and separated by 4.0 mm. The 3387-lead has four contacts (contact surface area, 5.985 mm²) spanning 10.5 mm. The individual contacts are 1.5 mm long and separated by 1.5 mm. (2) The EPA custom software application running on a tablet computer provides local computing and bidirectional connectivity between devices. (3) Cloud data and the analytics platform.

FIG. 5B illustrates continuous streaming of brain LFP enables monitoring of epileptiform activity [IES and seizure (Sz)] and Z. Bottom Circles show automated IES and seizure detections.

FIG. 5C illustrates representative raw impedance time series from HPC, AMG, and ANT recorded over multiple months from the subject living in natural home environment. Bottom, expanded time scales of average Z (z score with 2 h median filter) showing 24 h cycles over multiple days and weeks.

FIG. 6 illustrates a table presenting patient demographics and clinical information. In the table shown, the patients had mesial temporal lobe epilepsy with seizures (Szs) recorded from left and right HPC and AMG. All patients had drug-resistant epilepsy (DRE) with failure of at least two antiseizure medications (ASM). All patients had bilateral IES and Szs. The number of Szs and IESs were greater in the left compared with right HPC and AMG. M, Male; F, female; R, right; L, left; yrs, years; B, bilateral; RF, risk factors; TBI, traumatic brain injury; LOC, loss of consciousness; VNS, vagus nerve stimulation; TL, temporal lobe; FAS, focal aware seizures; FIAS, focal impaired awareness seizures; FTBC, focal to bilateral tonic-clonic seizures. The ASM medications include Cannabidiol (CBD), Carbamazepine (CBZ), Clonazepam (CLZ), Clorazepate (CZP), Cenobamate (CNB), Gabapentin (GBP), Lacosamide (LCM), Lamotrigine (LGT), Levetiracetam (LEV), Lorazepam (LZP), Oxcarbazepine (OXC), Phenytoin (PHT), and Valproic Acid (VPA).

FIG. 7 illustrates a table presenting electrode coregistration. Localization of 16 electrode contacts for subjects M1-5. The four leads were stereotactically targeted to left Thalamus (THL) (contacts E0, E1 E2 E3), left Amygdala-Hippocampus (AMG-HPC) (contacts E4, E5 E6 E7), right THL (contacts E8, E9 E10 E11), and right AMG-HPC (contacts E12, E13 E14 E15).

FIG. 8 illustrates a table presenting automated seizure detection and review results. A hypersensitive threshold was selected for the validated CNN-LSTM detector. The detector automatically detected 22,227 candidate electrographic seizures, and 2347 of the detected events were visually confirmed as electrographic seizures by human expert review.

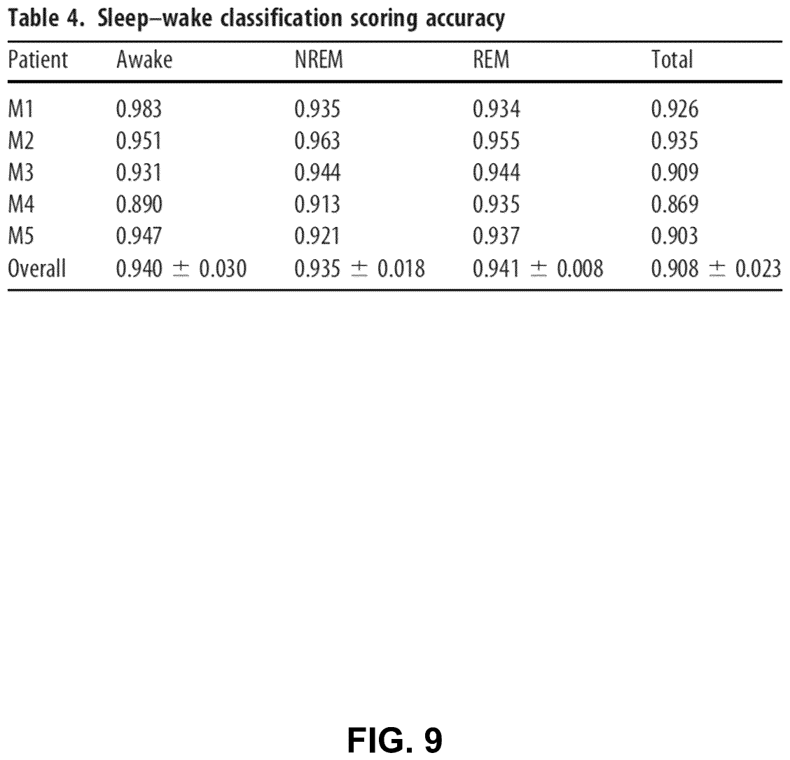

FIG. 9 illustrates a table presenting sleep-wake classification scoring accuracy. Results of automated sleep-wake state classification (F1 score) for awake, and REM, NREM sleep categories for subjects M1-5. The automated classifier uses LFP power-in-band inputs, and outputs are classification probabilities. The first hospital night was used for training, and the second hospital night for validation of the Naïve-Bayes classifier. The results reported here are from the third hospital night with simultaneous polysomnography and intracranial LFP recordings.

FIGS. 10A-C relate to impedance measurements in saline/microbead composites.

FIG. 10A illustrates a benchtop phantom fixture was used for testing two- and four-point impedance measurements in saline/microbead composites. For two-point monopolar measurements, current is injected into the sample medium using the 3387-lead contacts as the cathode (E1), and the anode is a large surface area contact (E0) at the bottom of the cylindrical container. The two-point measurement uses the same electrode contacts (E2 and E3 or E0) for both electrical stimulation and voltage sensing. The four-point impedance measurement uses different electrodes for stimulation (E1 and E4) and sensing (E3 and E2). The four-point measurements eliminate the interface electrode-electrolyte polarization, related to electrical stimulation, from the voltage measurements and provides a measurement of the medium.

FIG. 10B illustrates a graph for measured, the impedance measured using sinusoidal currents in saline and saline/microbead composites (1-4000 Hz). The two-point measurements are dominated at low frequency (<500 Hz) by the frequency-dependent capacitive double layer related to the electrolyte polarization at the electrode-electrolyte interface in both saline (red squares) and saline/microbead (blue squares). The four-point impedance measurement, using different electrodes for current injection and voltage response sensing, yields a purely resistive impedance with no frequency dependence (10-4000 Hz). The RC+S impedance measurement results are shown for comparison as a blue dashed line (saline/microbead) and red dashed line (saline).

FIG. 10C illustrates graphs for RC+S measurements. the RC+S calculates impedance using Ohm's law, Z=V/I, where I is the injected current (0.4 mA, 80 μs pulse width) and V is the voltage response measured at 70 μs. The voltage recording using two-point measurement shows the voltage response to the impulse current (0.4 mA, 80 μs pulse width) with charging of the electrode-electrolyte double layer capacitor, which reaches an asymptotic voltage within ˜50 μs. The RC+S impedance measurement can be seen to correlate with ˜1000 Hz sinusoidal current input.

FIG. 11 illustrates a sleep characteristics table. The average sleep onset times, offset times, number of NREM-REM cycles, and duration of NREM epoch are tabulated for all subjects. The average sleep duration for all five subjects is 08:56±(01:25) with 4:04±2.50 cycles. The average NREM epoch duration is 19.93±31.91 min. The table highlights the variability across subjects and within each subject.

FIG. 12A illustrates a circuit model for monopolar brain impedance measurements. The impedance contributions include the electrode-tissue interface, multiple brain compartments (ECS, vascular, cellular), body, and device-tissue impedance.

FIG. 12B illustrates example impedance traces from electrodes targeting HPC, AMG, and ANT anatomic locations within the first 4 weeks (˜30 d) postimplant. Postimplant impedance shows an increasing trend that asymptotes to a stable mean value after ˜14 d.

FIG. 12C illustrates normalized stable impedance for the anatomic locations. The stabilized Z values were estimated as the mean of the raw impedance between 15 and 21 d after implant and are scaled by multiplying by the electrode contact surface area. The normalized stable measures were aggregated across five subjects (M1-5) for each location. Error bar indicates ±1 SD. The asymptote values of AMG, ANT, and HPC impedance are similar when corrected for electrode surface area.

FIGS. 13A-B refer to temporal profiles of seizures and acute impedance changes.

FIG. 13A illustrate circular histograms illustrating the distribution of seizure onset times (red histograms) and sleep periods (gray filled) over a 24 h clock for each subject. The distribution of seizures as a function of time of day shows that seizures primarily occur during daytime and exhibit a bimodal distribution. A diurnal bimodal distribution of temporal lobe seizures was previously reported for temporal lobe epilepsy.

FIG. 13B illustrate impedance measurements from AMG, ANT, and HPC during the ±12 h surrounding spontaneous seizures (blue line) were compared with days without seizures (green line). The data demonstrate an increase in average impedance on days with seizures. Raw impedance times series were segmented to ±12 h around the seizure onset times. The mean value of the impedance was subtracted from each segment to obtain the normalized impedance. The normalized impedance time series were aggregated and smoothed using a 2.4 h moving window. For each subject, the smoothed normalized impedances were aggregated, and the mean value per anatomic location (ANT, AMG, HPC) was calculated. The red vertical line indicates the onset time of seizures (for blue traces) or surrogate segments without seizures within 24 h (green traces). The surrogate segments without seizures were selected to match the time-of-day temporal distribution of actual seizure events. The blue curve represents the mean value of seizure-related normalized impedance, incorporating all seizures, whereas the green curve represents the mean value of normalized impedance from 24 h segments without any seizures. The distribution of time of day of impedance data segments without seizures (green) is matched to the distribution of time of day of actual seizure events to account for baseline circadian changes in impedance. The shaded areas around the means indicate ±1 SEM, representing the uncertainty across the subjects (n=5).

FIGS. 14A-C relate to twenty-four-hour impedance cycles. Circular histograms of the impedance maximum (orange) and minimum (brown) are overlayed with average impedance (blue line) and sleep (shaded gray).

FIG. 14A illustrate circular histograms of representative of 24 h impedance values. Top left is a circular histogram of representative impedance maximum values. Bottom left, Circular histogram of representative of 24 h impedance minimum values from same subject (M1). The maximum impedance values occur in late daytime hours during wakefulness. The minimum impedance values are in the early morning hours during sleep. Top right, the running average impedance using a 4 h window with 2 h overlaps show the diurnal pattern with impedance increased during daytime and decreased during nighttime. Bottom middle, the average period of sleep determined from the automated sleep-wake classifier (gray filled). Right, Composite polar plot containing (1) minimum/maximum (min/max) impedance histograms, (2) average impedance, (3) sleep period.

FIG. 14B illustrate the group-level composite polar plots from HPC, ANT, and AMG averaged across all subjects demonstrates that the min/max impedance values over 24 h periods of day/night are not uniformly distributed.

FIG. 14C illustrate composite polar plots for individual subjects M1-5 over multiple months show the average impedance increases during wakefulness and decreases during sleep (blue curve). The HPC, AMG, and ANT histograms show the impedance reaches maximum values in the evening during wakefulness and minimum values during the night and early morning sleep before waking. Subject M2 displays less consistent 24 h cycles. The circular histogram distributions of the minimum (brown) and maximum (orange) values of the 24 h impedance cycle were tested using the circular Kuiper test and show strong phase preferences for night and day (*p, 0.05, **p, 0.01, ***p, 0.001). Subject M5 did not have a right AMG electrode because of a technical issue during surgery.

FIG. 15 illustrate a table representing circadian impedance cycles. The average time of minimum and maximum impedance values, and the magnitude of the minimum and maximum impedance difference is tabulated. All subjects had significant 24 h impedance cycles with the minimum impedance in the morning hours and maximum impedance in the evening hours. The statistically significant values in bold are for nonuniform circular distributions.

FIGS. 16A-D, refer to multiscale rhythms of human brain impedance. The AMG, ANT, and HPC impedance time series exhibit multiscale cycles with ultradian, circadian, and infradian periods.

FIG. 16A illustrates impedance time series and analysis. Top to bottom, Displays for the HPC, AMG, and ANT impedance time series and analysis. Top, Raw impedance time series. Middle, CWT of impedance time series demonstrating multiscale oscillations. The circadian period (1 d) is most prominent with little variability over the multiple-month record. Right, Amplitude index (gray) shows the high-power, narrow circadian peak that can be compared with the broad more diffuse ultradian and infradian bands. Bottom, Significance test for ultradian (yellow dots), circadian (red dots), and infradian (blue dots) cycles identified using the F score test.

FIG. 16B illustrate a blowup of CWT over a shorter evaluation window of 3 d provides a clearer visualization of ultradian oscillations (˜1.25-3 h) that are not visually evident in the long-term amplitude index and CWT.

FIG. 16C illustrates statistical testing using Thomson F test multitaper scheme to identify significant ultradian, circadian, and infradian impedance cycles. Top, the presence of cycles with ultradian and circadian periods was investigated using a 5 d moving window (z score and down sampled to 48 samples/day and moving step of 5 d). A 100 d moving window (moving step of 5 d) was used to investigate cycles in infradian band. Middle, Thomson F-test based on the multitaper scheme to test the periodicity at the FOIs. Bottom, the F-test was set at the level of the upper 99% (p=0.01) where the red dash on the scale color bar (right) indicates the F statistic above the critical value.

FIG. 16D illustrates box plots of percentage of significant impedance cycles in ANT, AMG, and HPC. The percentage of significant ultradian (1.0-1.5, 1.5-1.7, 1.7-4.8, and 4.8-21.6 h/cycle), circadian (0.9-1.1 d/cycle), and infradian (1.1-2.5, 2.5-10, 10-20, and 20-33 d/cycle) cycles was examined at each anatomic location under examination. The values were aggregated across subjects and present them as box plots at each location per period band. Note that the fundamental frequency of the sleep cycle (1.6 h/cycle or 5 cycles per 8 h) is centered at the ultradian band of 1.5-1.7 h/cycle. The shaded area highlights the period bands that have higher proportions of significant periodicity in addition to the circadian cycle.

FIGS. 17A-D relate to sleep-wake state dependence of human brain impedance. Limbic brain impedance depends on sleep-wake state.

FIG. 17A illustrates impedance changes over a single night during a hospital stay with gold standard sleep-wake classifications from polysomnography, including wakefulness and four cycles of NREM and REM sleep from subject M5. The impedance decreases during the transition from wakefulness to NREM sleep and increases during transitions from NREM to REM.

FIG. 17B illustrates group-level analysis (M1-5) shows that the average impedance in HPC, AMG, and ANT is lowest during NREM sleep, intermediate in REM sleep, and highest in wakefulness.

FIG. 17C illustrates group level analysis taken from impedance measurements during daytime naps. In subjects (M1-4) taking daytime naps including at least 30 min of NREM sleep, the HPC and AMG impedance is decreased in NREM sleep compared with wakefulness before the nap. These results during daytime naps further support that impedance cycles are related to dependence on the sleep-wake behavioral state.

FIG. 17D illustrates, at the individual subject level, the ANT, AMG, and HPC impedance is greater during wakefulness compared with NREM sleep in all subjects, and it is greater than REM sleep for all subjects except M5. The impedance in the ANT is greater in REM sleep compared with NREM for all subjects but only reaches significance in the HPC and AMG for M4. The impedance difference between wakefulness and REM sleep is smaller compared with the difference observed between NREM and wakefulness or NREM and REM sleep (*p, 0.05, **p, 0.01, ***p, 0.001; Mann-Whitney test with Bonferroni correction for multiple observations). AMG data was not available for M5.

FIG. 18 illustrates a table for impedance changes during sleep-wake behavioral state transitions. The table includes Average impedance change between wakefulness, NREM and REM sleep in the HPC, AMG, and ANT for the five subjects.

### Example Implementation 3

### Example Implementation 3 Introduction

Fast impedance changes, on millisecond timescales due to opening of ion channels in active neuron membrane, and slow impedance change, occurring over seconds to minutes attributed to cell swelling, have been observed during seizures. Very slow impedance changes occurring on timescales of minutes to hours are observed in the example implementation 3. This knowledge provides insights into the mechanism of seizure generation and be useful for seizure forecasting. In this example, seizure-related impedance change over 24 hours in humans with TLE were studied.

### Example Implementation 3 Methods

Five peoples with drug resistant TLE were implanted with bilateral electrodes in the thalamus, amygdala, and hippocampus. One patient was excluded from this analysis due to a prior epilepsy surgery resulting in inconsistent seizure-related impedance change. The impedance was sampled nonuniformly (every 5-15 minutes) over multiple months. Seizures were identified from both hemispheres, but mainly from the more epileptogenic left side. The distributions of seizure onset times were quantified with circular histograms. Surrogate data segments from days without seizures were generated and aligned to the same distributions of seizure onset as the data segments with seizures. Cross-correlation between impedance and seizures was estimated and compared to cross-correlation with surrogate seizures.

### Example Implementation 3 Results

The estimated cross-correlation showed a peak in correlation in the amygdala-hippocampus and posterior hippocampus. In left thalamus, the mean value of normalized impedance related to real seizures was lower than that related to surrogate seizures about 0-10 hours before seizure onset. In left amygdala-hippocampus, the mean value of normalized impedance related to real seizures was higher than that related to surrogate ones about 0-10 hours before seizure onset. Significant difference was found in the left side of posterior hippocampus. The normalized impedance related to real seizures was significantly higher (mean values were separated by >2 SEM) than that related to the surrogate seizures ˜0-4 hours before seizure onset.

### Example Implementation 3 Conclusion

The peaks of cross-correlated impedance may suggest that seizure onsets could be phase locked to the circadian cycle of impedance. The impedance of thalamus is likely lowered, while that of hippocampus elevated hours before the seizure onset. The significant increase of impedance 0-4 hours before seizure onset in the left posterior hippocampus is useful for forecasting seizure alert.

### Additional Example Data

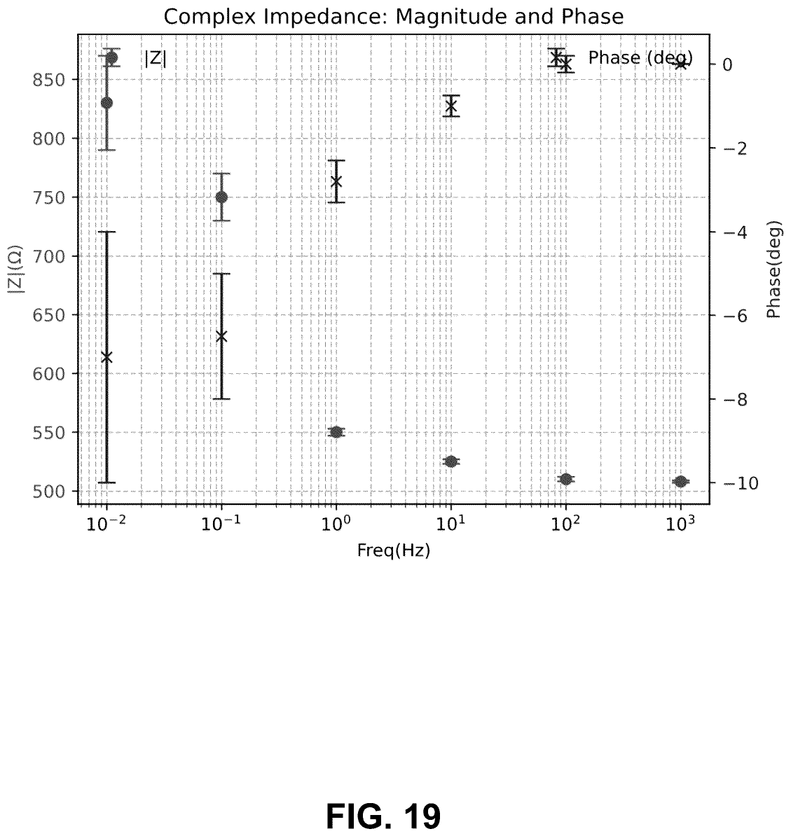

FIG. 19 illustrates a graph presenting four-point impedance measurement in a μbead-saline composite (30% bead vol+Saline). The data presented in FIG. 19 is an extension of the data presented in FIGS. 10A-C. Specifically, the data presented in FIG. 19 spans a greater frequency range. The graph shows the impedance magnitude (|Z|(ohm); red) and phase (Ø(degree); blue) over frequency 10⁻²-10³ (Hz). The impedance shows strong dispersion at low frequencies (e.g., less than ten hertz) with greater magnitude (e.g. 500 to 840 Ohms) and negative phase shift (e.g., zero to negative seven degrees). The dispersion of the impedance at low frequencies (e.g., less than ten Hz) is related to the same electrokinetic phenomena due to the double layer that forms around the beads in the saline-microbead composite. This same phenomena occurs in mammalian brain. This frequency dependence changes with seizures and as seizures approach with modification of the frequency dispersion can be used as a biomarker.

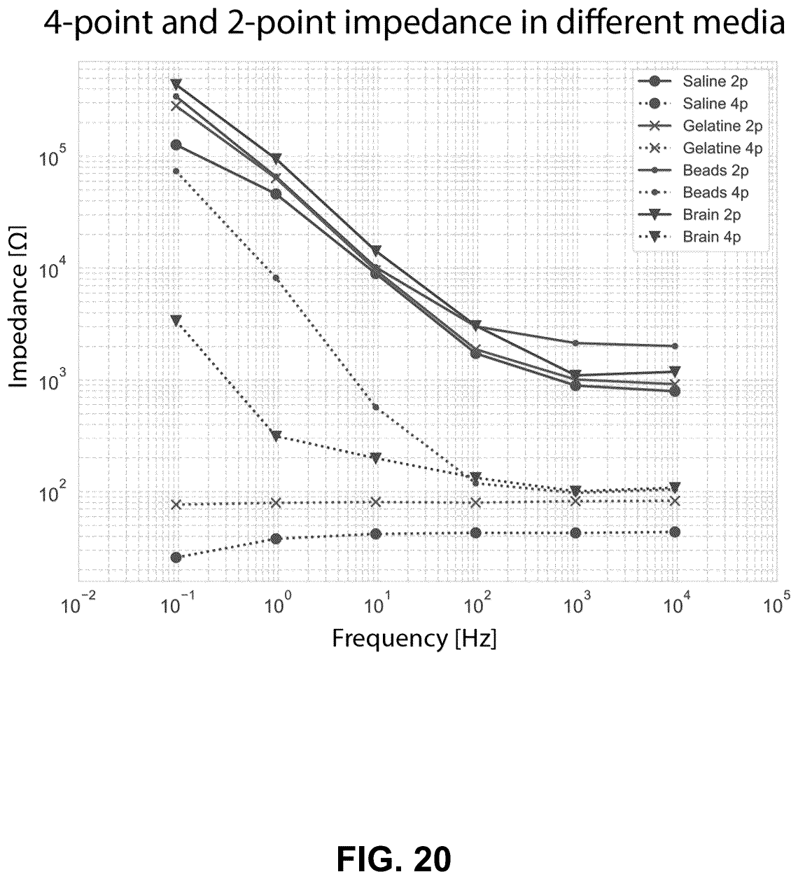

FIG. 20 illustrates a graph presenting an impedance (linear response) in saline, gelatine, saline-gelatine-μbead electrolytes, and in-vivo pig. All of the 2-point (2p) measurements show dispersion with frequency, even for saline alone, because of the electrolyte polarization effect at the stimulation/sensing electrode. The 4-point measurements (4p) show no dispersion for saline and gelatine alone. The 4-point measurements (4p) impedance measurement of pig brain and μbead-electrolyte composites shows strong dispersion with frequencies starting around or below 10-100 Hz.

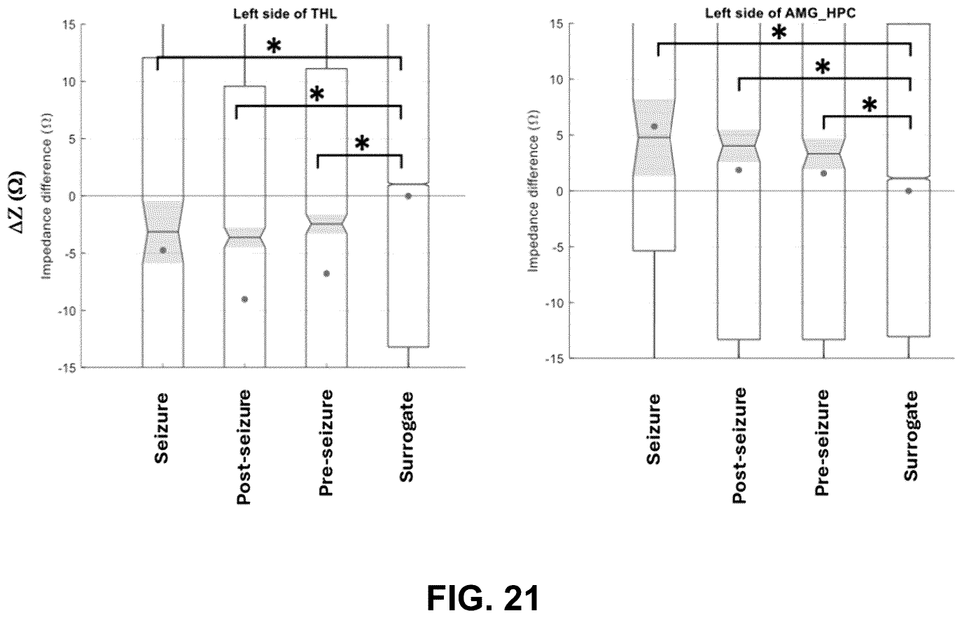

FIG. 21 illustrates graphs of measured impedance in human subjects. In this example, measurements from 5 human subjects are presented. Normalized human impedance (2-point 80 μs impulse response; 0.4 mA) in the thalamus (THL) (left) and amygdala-hippocampus (right) changes prior to hippocampal (HPC) seizures (5 minutes: Pre-seizure), during seizures (Seizure), and after seizures (5 minutes Post-seizure) compared to randomly sampled surrogate impedance values from the same time of day on days without seizures. The thalamus impedance decreases prior to the HPC seizure and HPC impedance increases.

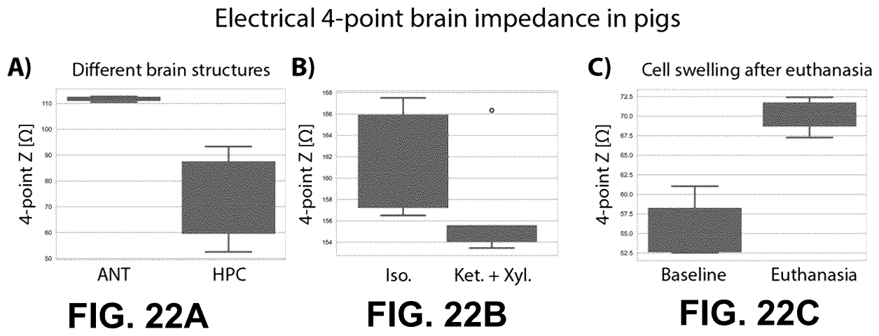

FIGS. 22A-C illustrate graphs for four-point measured impedance in-vivo pig. Each graph illustrates measurements with the penetrating electrodes targeting anterior nucleus of thalamus (ANT) and hippocampus (HPC) under different states. FIG. 22A shows the ANT and HPC under isoflurane anesthesia. This example shows the HPC impedance is lower than HPC. FIG. 22B shows the HPC impedance under isoflurane and ketamine/Xyzlazine (Ket.+Xyl). This example shows the impedance is lower under ketamine/Xylazine. FIG. 22C shows impedance during euthanasia with pentobarbital. This example shows the impedance increases with cell swelling.

In some implementations, the impedance describes the frequency dependent linear response voltage (0.001-10,000 Hz) generated by a test probe electric current. The impedance can be used a biomarker for cellular, extracellular matrix, and interstitial extracellular electrolyte fluid electrical interactions. The impedance frequency spectrum can be used to track behavioral state (sleep-wake), the glymphatic system function, and probability of seizure occurrence.

In some implementations, the temporal dynamics of impedance frequency dispersion (0.001-10,000 Hz) in different anatomical brain regions and their correlations are biomarkers are used for tracking behavioral state (sleep-wake), the glymphatic system function, and probability of seizure occurrence.

### Example Computing Devices

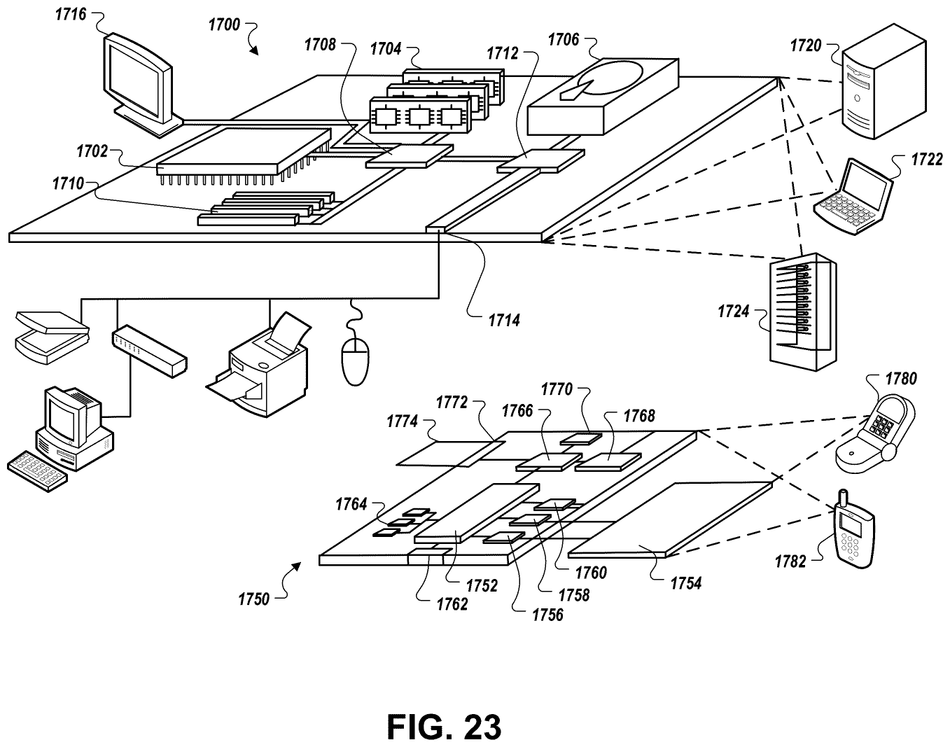

FIG. 23 shows an example of a computing device 1700 and a mobile computing device that can be used to implement the techniques described herein. The computing device 1700 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1700 includes a processor 1702, a memory 1704, a storage device 1706, a high-speed interface 1708 connecting to the memory 1704 and multiple high-speed expansion ports 1710, and a low-speed interface 1712 connecting to a low-speed expansion port 1714 and the storage device 1706. Each of the processor 1702, the memory 1704, the storage device 1706, the high-speed interface 1708, the high-speed expansion ports 1710, and the low-speed interface 1712, are interconnected using various busses, and may be mounted on a common motherboard or in other manners as appropriate. The processor 1702 can process instructions for execution within the computing device 1700, including instructions stored in the memory 1704 or on the storage device 1706 to display graphical information for a GUI on an external input/output device, such as a display 1716 coupled to the high-speed interface 1708. In other implementations, multiple processors and/or multiple buses may be used, as appropriate, along with multiple memories and types of memory. Also, multiple computing devices may be connected, with each device providing portions of the necessary operations (for example, as a server bank, a group of blade servers, or a multi-processor system).

The memory 1704 stores information within the computing device 1700. In some implementations, the memory 1704 is a volatile memory unit or units. In some implementations, the memory 1704 is a non-volatile memory unit or units. The memory 1704 may also be another form of computer-readable medium, such as a magnetic or optical disk.

The storage device 1706 is capable of providing mass storage for the computing device 1700. In some implementations, the storage device 1706 may be or contain a computer-readable medium, such as a floppy disk device, a hard disk device, an optical disk device, or a tape device, a flash memory or other similar solid state memory device, or an array of devices, including devices in a storage area network or other configurations. The computer program product may also contain instructions that, when executed, perform one or more methods, such as those described above. The computer program product can also be tangibly embodied in a computer- or machine-readable medium, such as the memory 1704, the storage device 1706, or memory on the processor 1702.

The high-speed interface 1708 manages bandwidth-intensive operations for the computing device 1700, while the low-speed interface 1712 manages lower bandwidth-intensive operations. Such allocation of functions is exemplary only. In some implementations, the high-speed interface 1708 is coupled to the memory 1704, the display 1716 (for example, through a graphics processor or accelerator), and to the high-speed expansion ports 1710, which may accept various expansion cards (not shown). In the implementation, the low-speed interface 1712 is coupled to the storage device 1706 and the low-speed expansion port 1714. The low-speed expansion port 1714, which may include various communication ports (for example, USB, Bluetooth, Ethernet, wireless Ethernet) may be coupled to one or more input/output devices, such as a keyboard, a pointing device, a scanner, or a networking device such as a switch or router, for example, through a network adapter.

The computing device 1700 may be implemented in a number of different forms, as shown in the figure. For example, it may be implemented as a standard server 1720, or multiple times in a group of such servers. In addition, it may be implemented in a personal computer such as a laptop computer 1722. It may also be implemented as part of a rack server system 1724. Alternatively, components from the computing device 1700 may be combined with other components in a mobile device (not shown), such as a mobile computing device 1750. Each of such devices may contain one or more of the computing device 1700 and the mobile computing device 1750, and an entire system may be made up of multiple computing devices communicating with each other.

The mobile computing device 1750 includes a processor 1752, a memory 1764, an input/output device such as a display 1754, a communication interface 1766, and a transceiver 1768, among other components. The mobile computing device 1750 may also be provided with a storage device, such as a micro-drive or other device, to provide additional storage. Each of the processor 1752, the memory 1764, the display 1754, the communication interface 1766, and the transceiver 1768, are interconnected using various buses, and several of the components may be mounted on a common motherboard or in other manners as appropriate.

The processor 1752 can execute instructions within the mobile computing device 1750, including instructions stored in the memory 1764. The processor 1752 may be implemented as a chipset of chips that include separate and multiple analog and digital processors. The processor 1752 may provide, for example, for coordination of the other components of the mobile computing device 1750, such as control of user interfaces, applications run by the mobile computing device 1750, and wireless communication by the mobile computing device 1750.

The processor 1752 may communicate with a user through a control interface 1758 and a display interface 1756 coupled to the display 1754. The display 1754 may be, for example, a TFT (Thin-Film-Transistor Liquid Crystal Display) display or an OLED (Organic Light Emitting Diode) display, or other appropriate display technology. The display interface 1756 may comprise appropriate circuitry for driving the display 1754 to present graphical and other information to a user. The control interface 1758 may receive commands from a user and convert them for submission to the processor 1752. In addition, an external interface 1762 may provide communication with the processor 1752, so as to enable near area communication of the mobile computing device 1750 with other devices. The external interface 1762 may provide, for example, for wired communication in some implementations, or for wireless communication in other implementations, and multiple interfaces may also be used.

The memory 1764 stores information within the mobile computing device 1750. The memory 1764 can be implemented as one or more of a computer-readable medium or media, a volatile memory unit or units, or a non-volatile memory unit or units. An expansion memory 1774 may also be provided and connected to the mobile computing device 1750 through an expansion interface 1772, which may include, for example, a SIMM (Single In Line Memory Module) card interface. The expansion memory 1774 may provide extra storage space for the mobile computing device 1750, or may also store applications or other information for the mobile computing device 1750. Specifically, the expansion memory 1774 may include instructions to carry out or supplement the processes described above, and may include secure information also. Thus, for example, the expansion memory 1774 may be provide as a security module for the mobile computing device 1750, and may be programmed with instructions that permit secure use of the mobile computing device 1750. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. The computer program product contains instructions that, when executed, perform one or more methods, such as those described above. The computer program product can be a computer- or machine-readable medium, such as the memory 1764, the expansion memory 1774, or memory on the processor 1752. In some implementations, the computer program product can be received in a propagated signal, for example, over the transceiver 1768 or the external interface 1762.

The mobile computing device 1750 may communicate wirelessly through the communication interface 1766, which may include digital signal processing circuitry where necessary. The communication interface 1766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1768 using a radio-frequency. In addition, short-range communication may occur, such as using a Bluetooth, WiFi, or other such transceiver (not shown). In addition, a GPS (Global Positioning System) receiver module 1770 may provide additional navigation- and location-related wireless data to the mobile computing device 1750, which may be used as appropriate by applications running on the mobile computing device 1750.

The mobile computing device 1750 may also communicate audibly using an audio codec 1760, which may receive spoken information from a user and convert it to usable digital information. The audio codec 1760 may likewise generate audible sound for a user, such as through a speaker, for example, in a handset of the mobile computing device 1750. Such sound may include sound from voice telephone calls, may include recorded sound (e.g., voice messages, music files, etc.) and may also include sound generated by applications operating on the mobile computing device 1750.

The mobile computing device 1750 may be implemented in a number of different forms, as shown in the figure. For example, it may be implemented as a cellular telephone 1780. It may also be implemented as part of a smart-phone 1782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### EXAMPLE IMPLEMENTATIONS

Some of the examples described herein include or are defined by the following implementations.

Implementation A1 is a method for predicting and managing seizures in an individual, the method comprising obtaining at least one measurement of an impedance of brain tissue of the individual to determine an impedance signature of the brain tissue of the individual, processing the impedance signature of the brain tissue to predict whether the individual is likely to experience a seizure, and in response to predicting based on the impedance signature that the individual is likely to experience a seizure, initiating a remedial action that comprises at least one of alerting the individual or another user that the individual is likely to experience the seizure, applying an electrical stimulus to the brain tissue of the individual to reduce a likelihood of the seizure occurring or to reduce a severity of the seizure, or delivering a drug to the brain tissue of the individual to reduce a likelihood of the seizure occurring or to reduce a severity of the seizure.

Implementation A2 is the method of implementation A1, wherein sampling the impedance of the brain tissue of an individual includes: electrically stimulating the brain tissue with an implanted device that injects an electrical current into the brain tissue, and sensing a voltage in the brain tissue with the implanted device in response electrically stimulating the brain tissue, and calculating the impedance based on the electrical current and the sensed voltage.

Implementation A3 is the method of any of implementations A1 and A2, wherein a machine learning model is used to predict whether the individual is likely to experience a seizure.

Implementation A4 is the method of implementation A3, wherein the machine learning model is an unsupervised model such as a deep learning model

Implementation A5 is the method of any of implementations A3 and A4, wherein the machine learning algorithm is a support vector machine (SVM) or random forrest (RF).

Implementation A6 is the method of any of implementations A3-A5, wherein the machine learning model outputs the probability of an upcoming seizure, wherein the remedial action is initiated when the probability of the upcoming seizure exceeds a threshold.

Implementation A7 is the method of any of implementations A3-A6, wherein the machine learning model is a deep learning model.

Implementation A8 is the method of implementation A7, wherein the deep learning model is a bi-directional long-term memory model.

Implementation A9 is the method of any of implementations A7 and A8, wherein training the deep learning model comprises aggregating model inputs by segmenting impedance data and determining rate of impedance change and time-frequency power signals for each segment, processing the model inputs with a parallel 1-D convolution network (CNN) to extract features, constructing feature representations to learn valid information through forward and/or backward propagation of the extracted features, applying an attention mechanism to redistribute weights of the feature representations to emphasize critical features of preictal stage, and training a full connection layer to output a preictal probability based on the feature representations with redistributed weights.

Implementation A10 is the method of implementation A9, wherein the preictal probability is compared to a threshold to determine whether a seizure is likely to occur.

Implementation A11 is the method of any of implementations A7-A10, wherein the deep learning model is trained using a cloud computing system.

Implementation A12 is the method of any of implementations A1-A11, wherein processing the impedance signature of the brain tissue to predict whether the individual is likely to experience a seizure is further based on a detected brain state.

Implementation A13 is the method of implementation A12, wherein brain states include an awake state and one or more sleep states.

Implementation A14 is the method of any implementations A1-A13, the method further comprising measuring glymphatic stages of the brain tissue based on the impedance signature, and determining levels of sleep stages for the individual based on the measured glymphatic stages.

Implementation A15 is the method of any of implementations A1-A14, wherein the impedance signature includes an absolute value of observed impedance and a rate of change in impedance.

Implementation A16 is the method of any of implementations A1-A15, wherein prior altering the individual, sending the impedance signature to a monitoring station for review by a provider individual.

Implementation A17 is the method of any of implementations A1-16, wherein the individual is a mammal.

Implementation A18 is the method of any of implementations A1-A17, wherein the individual is a human.

Implementation A19 is the method of any of implementations A1-A18, wherein the impedance signature is based on measurements of impedance at a single location in a brain of the individual.

Implementation A20 is the method of any of implementations A1-A19, wherein the impedance signature is based on measurements of impedance at multiple locations in a brain of the individual.

Implementation A21 is the method of any of implementations A1-A20, wherein the impedance signature is based on measurements of impedance spanning a time interval of at least 1, 2, 3, 4, or 5 hours.

Implementation A22 is the method of any of implementations A1-A21, wherein the impedance signature is based on measurements of impedance spanning a time interval of at least 0.5, 1, 2, 3, 4, or 5 days.

Implementation A23 is the method of any implementations A1-A22, wherein the impedance signature is based on measurements of impedance spanning a time interval of at least 20-33 days.

Implementation A24 is the method of any of implementations A1-A23, wherein processing the impedance signature to predict whether the individual is likely to experience a seizure comprises comparing the impedance signature to a model impedance signature.

Implementation A25 is the method of implementation A24, wherein the model impedance signature is personalized to the individual based on recordings from the individual.

Implementation A26 is the method of any implementations A24 and A25, wherein the model impedance signature comprises a threshold impedance level, and processing the impedance signature comprises determining that at least one measurement of the impedance of the individual exceeds the threshold impedance level.

Implementation A27 is the method of implementation A26, wherein the threshold impedance level is adjusted based on a detected brain state of the individual, wherein the detected brain state is selected from an awake state, a rapid eye movement (REM) sleep state, a non-rapid eye movement (NREM) sleep state and a NREM category including drowsiness (N1), sleep (N2), and deep sleep (N3) or microstates within these NREM sleep categories.

Implementation A28 is the method of any of implementations A26 and A27, wherein the threshold impedance level is adjusted based on an ultradian rhythm of the individual, a circadian rhythm of the individual, or an infradian rhythm of the individual.

Implementation A29 is the method of any of implementations A24-A28, wherein the model impedance signature comprises a threshold rate of change of impedance, and processing the impedance signature comprises determining that a rate of change of impedance of the brain tissue of the individual is greater than the threshold rate of change of impedance indicated by the model impedance signature.

Implementation A30 is the method of implementation A29, wherein the threshold rate of change of impedance is adjusted based on a detected brain state of the individual, wherein the detected brain state is selected from an awake state, a rapid eye movement (REM) sleep state, a non-rapid eye movement (NREM) sleep state and a NREM category including drowsiness (N1), sleep (N2), and deep sleep (N3) or microstates within these NREM sleep categories.

Implementation A31 is the method of any of implementations A29 and A30, wherein the threshold rate of change of impedance is adjusted based on an ultradian rhythm of the individual, a circadian rhythm of the individual, or an infradian rhythm of the individual.

Implementation A32 is the method of any of implementations A1-A31, wherein the impedance describes a frequency dependent linear response voltage generated by a test probe electric current.

Implementation A33 is the method of implementation A32, wherein a frequency of the frequency dependent linear response voltage is between 0.001 hertz and 10,000 hertz.

Implementation A34 is the method of any of implementations A1-A33, wherein the impedance is used as a biomarker for at least one of: (a) cellular electrical interactions; (b) extracellular matrix electrical interactions; (c) interstitial extracellular electrolyte fluid electrical interactions; or (d) any combination of (a), (b), and (c).

Implementation A35 is the method of any of implementations A1-A34, wherein an impedance frequency spectrum is constructed using the at least one measurement of the impedance of the brain tissue.

Implementation A36 is the method of implementation A35, wherein the impedance frequency spectrum is used to track: a behavioral state of the individual; (b) a glymphatic system function of the individual; (c) a probability of seizure occurrence; or (d) any combination of (a), (b), and (c).

Implementation A37 is the system method any of implementations A1-A36, wherein temporal dynamics and correlations of impedance frequency dispersion in different anatomical brain regions are biomarkers used to track: (a) a behavioral state of the individual; (b) a glymphatic system function of the individual; (c) a probability of seizure occurrence; or (d) any combination of (a), (b), and (c).

Implementation A38 is the method of implementation A37, wherein the temporal dynamics and correlations of frequency dispersion are detected between 0.001 hertz and 10,000 hertz.

Implementation B1 is a system comprising an implanted device configured to capture at least one measurement of an impedance of brain tissue of an individual and a computing device configured to obtain, from the implanted device, the at least one measurement of the impedance of the brain tissue of the individual and in response to a prediction that the individual is likely to experience a seizure, initiate a remedial action that comprises at least one of alerting the individual or another user that the individual is likely to experience the seizure, applying an electrical stimulus to the brain tissue of the individual to reduce a likelihood of the seizure occurring or to reduce a severity of the seizure, or delivering a drug to the brain tissue of the individual to reduce a likelihood of the seizure occurring or to reduce a severity of the seizure, wherein the prediction that the individual is likely to experience a seizure is determined by processing an impedance signature determined from the at least one measurement of the impedance of the brain tissue of the individual.

Implementation B2 is the system of implementation B1, wherein the computing device operates an application that is configured to receive an individual input for indicating that the individual is experiencing a seizure or recently experienced a seizure.

Implementation B3 is the system of implementation B2, wherein the individual input is used to determine when the individual experience a seizure and impedance data from around a time the seizure occurred is collected and processed to determine an impedance signature that corresponds to the individual being likely to experience a seizure.

Implementation B4 is the system of any of implementations B1-B3, wherein the prediction is based on a detected change in impedance as compared to a threshold.

Implementation B5 is the system of implementation B4, wherein the threshold is patient specific.

Implementation B6 is the system of any of implementations B1-B5, wherein the alert is presented to an individual ten minutes before the seizure is expected to occur.

Implementation B7 is the system of any of implementations B1-B6, wherein the implantable device is configured to receive a two-point impedance measurement.

Implementation B8 is the system of any of implementations B1-B7, wherein the implantable device is configured to receive a four-point impedance measurement.

Implementation B9 is the system of any of implementations B1-B8, wherein the impedance describes a frequency dependent linear response voltage generated by a test probe electric current.

Implementation B10 is the system of implementation B9, wherein a frequency of the frequency dependent linear response voltage is between 0.001 hertz and 10,000 hertz.

Implementation B11 is the system of any of implementations B1-B10, wherein the impedance is used as a biomarker for at least one of: (a) cellular electrical interactions; (b) extracellular matrix electrical interactions; (c) interstitial extracellular electrolyte fluid electrical interactions; or (d) any combination of (a), (b), and (c).

Implementation B12 is the system of any of implementations B1-B11, wherein an impedance frequency spectrum is constructed using the at least one measurement of the impedance of the brain tissue.

Implementation B13 is the system of implementation B12, wherein the impedance frequency spectrum is used to track: a behavioral state of the individual; (b) a glymphatic system function of the individual; (c) a probability of seizure occurrence; or (d) any combination of (a), (b), and (c).

Implementation B14 is the system of any of implementations B1-B13, wherein temporal dynamics and correlations of impedance frequency dispersion in different anatomical brain regions are biomarkers used to track: (a) a behavioral state of the individual; (b) a glymphatic system function of the individual; (c) a probability of seizure occurrence; or (d) any combination of (a), (b), and (c).

Implementation B15 is the system of implementation B14, wherein the temporal dynamics and correlations of frequency dispersion are detected between 0.001 hertz and 10,000 hertz.

In situations in which the systems, methods, devices, and other techniques here collect personal information (e.g., context data) about users, or may make use of personal information, the users may be provided with an opportunity to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

Although various implementations have been described in detail above, other modifications are possible. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A method for predicting and managing seizures in an individual, the method comprising:
obtaining at least one measurement of an impedance of brain tissue of the individual to determine an impedance signature of the brain tissue of the individual;
processing the impedance signature of the brain tissue to predict whether the individual is likely to experience a seizure; and
in response to predicting based on the impedance signature that the individual is likely to experience a seizure, initiating a remedial action that comprises at least one of alerting the individual or another user that the individual is likely to experience the seizure, applying an electrical stimulus to the brain tissue of the individual to reduce a likelihood of the seizure occurring or to reduce a severity of the seizure, or delivering a drug to the brain tissue of the individual to reduce a likelihood of the seizure occurring or to reduce a severity of the seizure.

2. The method of claim 1, wherein sampling the impedance of the brain tissue of an individual includes:
electrically stimulating the brain tissue with an implanted device that injects an electrical current into the brain tissue;
sensing a voltage in the brain tissue with the implanted device in response electrically stimulating the brain tissue; and
calculating the impedance based on the electrical current and the sensed voltage.

3. The method of claim 1, wherein a machine learning model is used to predict whether the individual is likely to experience a seizure.

4. The method of claim 3, wherein the machine learning model is an unsupervised model such as a deep learning model.

5. The method of claim 3, wherein the machine learning model is a support vector machine (SVM) or random forrest (RF).

6. The method of claim 3, wherein the machine learning model outputs a probability of an upcoming seizure, wherein the remedial action is initiated when the probability of the upcoming seizure exceeds a threshold.

7. The method of claim 3, wherein the machine learning model is a deep learning model.

8. The method of claim 7, wherein the deep learning model is a bi-directional long-term memory model.

9. The method of claim 7, wherein training the deep learning model comprises:
aggregating model inputs by segmenting impedance data and determining rate of impedance change and time-frequency power signals for each segment;
processing the model inputs with a parallel 1-D convolution network (CNN) to extract features;
constructing feature representations to learn valid information through forward and/or backward propagation of the extracted features;
applying an attention mechanism to redistribute weights of the feature representations to emphasize critical features of preictal stage; and
training a full connection layer to output a preictal probability based on the feature representations with redistributed weights.

10. The method of claim 9, wherein the preictal probability is compared to a threshold to determine whether a seizure is likely to occur.

11. The method of claim 7, wherein the deep learning model is trained using a cloud computing system.

12. The method of claim 1, wherein processing the impedance signature of the brain tissue to predict whether the individual is likely to experience a seizure is further based on a detected brain state.

13. The method of claim 12, wherein brain states include an awake state and one or more sleep states.

14. The method of claim 1, the method further comprising:
measuring glymphatic stages of the brain tissue based on the impedance signature; and
determining levels of sleep stages for the individual based on the measured glymphatic stages.

15. The method of claim 1, wherein the impedance signature includes an absolute value of observed impedance and a rate of change in impedance.

16. The method of claim 1, wherein prior altering the individual, sending the impedance signature to a monitoring station for review by a provider individual.

17. The method of claim 1, wherein the individual is a mammal.

18. The method of claim 1, wherein the individual is a human.

19. The method of claim 1, wherein the impedance signature is based on measurements of impedance at a single location in a brain of the individual.

20. The method of claim 1, wherein the impedance signature is based on measurements of impedance at multiple locations in a brain of the individual.

21. The method of claim 1, wherein the impedance signature is based on measurements of impedance spanning a time interval of at least 1, 2, 3, 4, or 5 hours.

22. The method of claim 1, wherein the impedance signature is based on measurements of impedance spanning a time interval of at least 0.5, 1, 2, 3, 4, or 5 days.

23. The method of claim 1, wherein the impedance signature is based on measurements of impedance spanning a time interval of at least 20-33 days.

24. The method of claim 1, wherein processing the impedance signature to predict whether the individual is likely to experience a seizure comprises comparing the impedance signature to a model impedance signature.

25. The method of claim 24, wherein the model impedance signature is personalized to the individual based on recordings from the individual.

26. The method of claim 24, wherein the model impedance signature comprises a threshold impedance level, and processing the impedance signature comprises determining that at least one measurement of the impedance of the individual exceeds the threshold impedance level.

27. The method of claim 26, wherein the threshold impedance level is adjusted based on a detected brain state of the individual, wherein the detected brain state is selected from an awake state, a rapid eye movement (REM) sleep state, a non-rapid eye movement (NREM) sleep state and a NREM category including drowsiness (N1), sleep (N2), and deep sleep (N3) or microstates within these NREM sleep categories.

28. The method of claim 26, wherein the threshold impedance level is adjusted based on an ultradian rhythm of the individual, a circadian rhythm of the individual, or an infradian rhythm of the individual.

29. The method of claim 24, wherein the model impedance signature comprises a threshold rate of change of impedance, and processing the impedance signature comprises determining that a rate of change of impedance of the brain tissue of the individual is greater than the threshold rate of change of impedance indicated by the model impedance signature.

30. The method of claim 29, wherein the threshold rate of change of impedance is adjusted based on a detected brain state of the individual, wherein the detected brain state is selected from an awake state, a rapid eye movement (REM) sleep state, a non-rapid eye movement (NREM) sleep state and a NREM category including drowsiness (N1), sleep (N2), and deep sleep (N3) or microstates within these NREM sleep categories.

31. The method of claim 29, wherein the threshold rate of change of impedance is adjusted based on an ultradian rhythm of the individual, a circadian rhythm of the individual, or an infradian rhythm of the individual.

32. A system comprising:
an implanted device configured to capture at least one measurement of an impedance of brain tissue of an individual; and
a computing device configured to:
obtain, from the implanted device, the at least one measurement of the impedance of the brain tissue of the individual; and
in response to a prediction that the individual is likely to experience a seizure, initiate a remedial action that comprises at least one of alerting the individual or another user that the individual is likely to experience the seizure, applying an electrical stimulus to the brain tissue of the individual to reduce a likelihood of the seizure occurring or to reduce a severity of the seizure, or delivering a drug to the brain tissue of the individual to reduce a likelihood of the seizure occurring or to reduce a severity of the seizure,
wherein the prediction that the individual is likely to experience a seizure is determined by processing an impedance signature determined from the at least one measurement of the impedance of the brain tissue of the individual.

33. The system of claim 32, wherein the computing device operates an application that is configured to receive an individual input for indicating that the individual is experiencing a seizure or recently experienced a seizure.

34. The system of claim 33, wherein the individual input is used to determine when the individual experience a seizure and impedance data from around a time the seizure occurred is collected and processed to determine an impedance signature that corresponds to the individual being likely to experience a seizure.

35. The system of claim 32, wherein the prediction is based on a detected change in impedance as compared to a threshold.

36. The system of claim 35, wherein the threshold is patient specific.

37. The system of claim 32, wherein alerting the individual or another user that the individual is likely to experience the seizure is provided ten minutes before the seizure is expected to occur.

38. The system of claim 32, wherein the implantable device is configured to receive a two-point impedance measurement.

39. The system of claim 32, wherein the implantable device is configured to receive a four-point impedance measurement.

40. The system of claim 32, wherein the impedance describes a frequency dependent linear response voltage generated by a test probe electric current.

41. The system of claim 40, wherein a frequency of the frequency dependent linear response voltage is between 0.001 hertz and 10,000 hertz.

42. The system of claim 32, wherein the impedance is used as a biomarker for at least one of:
(a) cellular electrical interactions;
(b) extracellular matrix electrical interactions;
(c) interstitial extracellular electrolyte fluid electrical interactions; or
(d) any combination of (a), (b), and (c).

43. The system of claim 32, wherein an impedance frequency spectrum is constructed using the at least one measurement of the impedance of the brain tissue.

44. The system of claim 43, wherein the impedance frequency spectrum is used to track:
(a) a behavioral state of the individual;
(b) a glymphatic system function of the individual;
(c) a probability of seizure occurrence; or
(d) any combination of (a), (b), and (c).

45. The system of claim 32, wherein temporal dynamics and correlations of impedance frequency dispersion in different anatomical brain regions are biomarkers used to track:
(a) a behavioral state of the individual;
(b) a glymphatic system function of the individual;
(c) a probability of seizure occurrence; or
(d) any combination of (a), (b), and (c).

46. The system of claim 45, wherein the temporal dynamics and correlations of frequency dispersion are detected between 0.001 hertz and 10,000 hertz.
